(19) United States
(12) Patent Application Publication
(10) Pub. No.: US 2015/0328310 A1
(43) Pub. Date: Nov. 19, 2015

(54) METHODS AND COMPOSITIONS RELATING TO TREATMENT OF CANCER

(71) Applicants: Joshua E. ALLEN, Philadelphia, PA (US); Wifik S. EL-DEIRY, Bryn Mawr, PA (US); Arun K. SHARMA, Hummelstown, PA (US); Rosalyn IRBY, Hummelstown, PA (US); THE PENN STATE RESEARCH FOUNDATION, University Park, PA (US)

(72) Inventors: Joshua E. Allen, Philadelphia, PA (US); Wafik S. El-Deiry, Bryn Mawr, PA (US); Arun K. Sharma, Hummelstown, PA (US); Shantu G. Amin, Union City, NJ (US)

(21) Appl. No.: 14/652,363

(22) PCT Filed: Dec. 20, 2013
(86) PCT No.: PCT/US2013/076869

§ 371 Date: Jun. 15, 2015

Related U.S. Application Data
(60) Provisional application No. 61/740,126, filed on Dec. 20, 2012.

(51) Int. Cl. A61K 39/395; A61K 31/7028; A61K 31/095

(52) U.S. Cl. CPC A61K 39/3955; A61K 31/095; A61K 31/7028; A61K 2039/505

(57) ABSTRACT

Compositions and methods for treating cancer in a subject in need thereof are provided according to aspects of the present disclosure which include both cetuximab and ISC-4, as a combination formulation or as separate formulations. Methods of treating cancer in a subject in need thereof are provided according to aspects of the present disclosure wherein the subject has cancer characterized by wild-type KRAS wherein the methods include administering a combination of cetuximab and ISC-4 as a combination formulation or separately, and wherein administration of the combination of cetuximab and ISC-4 provides a synergistic anti-cancer effect, treating the cancer.

### REFERENCE TO RELATED APPLICATION

This application claims priority from U.S. Provisional Patent Application Ser. No. 61/740,126, filed Dec. 20, 2012, the entire content of which is incorporated herein by reference.

### GOVERNMENT SUPPORT

This invention was made with government support under Grant No. CA143999, awarded by the National Institutes of Health. The Government has certain rights in the invention.

### FIELD OF THE INVENTION

Methods and compositions for treatment of cancer in a subject in need thereof are provided according to general aspects of the present invention. Methods and compositions for treatment of cancer in a subject in need thereof are provided according to specific aspects of the present invention which include administering both cetuximab and ISC-4, as a combination formulation or as separate formulations.

### BACKGROUND OF THE INVENTION

There is a continuing need for methods and compositions for treatment of cancer in a subject in need thereof. Such methods and compositions are provided according to the present invention.

### SUMMARY OF THE INVENTION

Methods of treating cancer in a subject in need thereof are provided according to aspects of the present invention which include administering both cetuximab and ISC-4, as a combination formulation or as separate formulations.

Methods of treating cancer in a subject in need thereof are provided according to aspects of the present invention which include administering a combination of cetuximab and ISC-4 as a combination formulation or separately, wherein the cancer is characterized by wild-type KRAS.

Methods of treating cancer in a subject in need thereof are provided according to aspects of the present invention which include administering a combination of cetuximab and ISC-4 as a combination formulation or separately, wherein the cancer is characterized by wild-type KRAS such that the wild-type KRAS does not have an activating KRAS mutation.

Methods of treating cancer in a subject in need thereof are provided according to aspects of the present invention which include administering a combination of cetuximab and ISC-4 as a combination formulation or separately, wherein the cancer is characterized by wild-type KRAS such that the wild-type KRAS does not have an activating KRAS mutation, in codon 12, 13 or 61, with reference to human KRAS.

Methods of treating cancer in a subject in need thereof are provided according to aspects of the present invention which include administering a combination of cetuximab and ISC-4 as a combination formulation or separately, wherein the cancer is characterized by wild-type KRAS such that the wild-type KRAS does not have activating KRAS mutations Q61H, G12S, G12V, G12A or G13D, with reference to human KRAS.

Methods of treating cancer in a subject in need thereof are provided by the present invention which include administering a combination of cetuximab and ISC-4 as a combination formulation or separately, wherein the cancer is colorectal cancer characterized by wild-type KRAS.

Methods of treating colorectal cancer in a subject in need thereof are provided according to aspects of the present invention which include administering a combination of cetuximab and ISC-4 as a combination formulation or separately, wherein the colorectal cancer is characterized by wild-type KRAS such that the wild-type KRAS does not have an activating KRAS mutation.

Methods of treating cancer in a subject in need thereof are provided according to aspects of the present invention which include administering a combination of cetuximab and ISC-4 as a combination formulation or separately, wherein the cancer is colorectal cancer characterized by wild-type KRAS such that the wild-type KRAS does not have an activating KRAS mutation, in codon 12, 13 or 61, with reference to human KRAS.

Methods of treating cancer in a subject in need thereof are provided according to aspects of the present invention which include administering a combination of cetuximab and ISC-4 as a combination formulation or separately, wherein the cancer is colorectal cancer characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include obtaining a first sample containing or suspected of containing cancer cells from the subject prior to administering the combination of cetuximab and ISC-4; administering a combination of cetuximab and ISC-4 as a combination formulation or separately, obtaining a second sample containing or suspected of containing cancer cells from the subject after administering the combination of cetuximab and ISC-4; and assaying the first and second samples for one or more markers of apoptosis, wherein increased apoptosis in the second sample compared to the first sample indicates therapeutic activity of the administered cetuximab and ISC-4, thereby monitoring effectiveness of administering the combination of cetuximab and ISC-4. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation, in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS. According to aspects of such methods, the cancer is colorectal cancer characterized by wild-type KRAS, such that the wild-type KRAS does not have an activating KRAS mutation. According to aspects of such methods, the cancer is colorectal cancer characterized by wild-type KRAS such that the wild-type KRAS does not have an activating KRAS mutation, in codon 12, 13 or 61, with reference to human KRAS. According to aspects of such methods, the cancer is colorectal cancer characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include obtaining a first sample containing or suspected of containing cancer cells from the subject prior to administering the combination of cetuximab and ISC-4; administering a combination of cetuximab and ISC-4 as a combination formulation or separately; obtaining a second sample containing or suspected of containing cancer cells from the subject after administering the combination of cetuximab and ISC-4; and assaying the first and second samples for phospho-Akt, wherein decreased phospho-Akt in the second sample compared to the first sample indicates therapeutic activity of the administered cetuximab and ISC-4, thereby monitoring effectiveness of administering the combination of cetuximab and ISC-4. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation, in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS. According to aspects of such methods, the cancer is colorectal cancer characterized by wild-type KRAS, such that the wild-type KRAS does not have an activating KRAS mutation. According to aspects of such methods, the cancer is colorectal cancer characterized by wild-type KRAS such that the wild-type KRAS does not have an activating KRAS mutation, in codon 12, 13 or 61, with reference to human KRAS. According to aspects of such methods, the cancer is colorectal cancer characterized by wild-type KRAS such that the wild-type KRAS does not have activating KRAS mutations Q61H, G12S, G12V, G12A or G13D, with reference to human KRAS.

According to aspects of methods of treating cancer of the present invention, the cetuximab and ISC-4 are administered simultaneously or sequentially. In a non-limiting example, the cetuximab and ISC-4 are administered sequentially within a period of time selected from: one hour, two hours, four hours, eight hours, twelve hours and twenty-four hours.

Pharmaceutical compositions are provided according to aspects of the present invention which include both cetuximab and ISC-4.

Commercial packages are provided according to aspects of the present invention which include both cetuximab and ISC-4, wherein the cetuximab and ISC-4 are provided as a single pharmaceutical formulation or as separate pharmaceutical formulations.

Methods of treating cancer in a subject in need thereof are provided by the present invention which include administering a combination of cetuximab and an ISC-4 prodrug in combination or separately.

Methods of treating cancer in a subject in need thereof are provided according to aspects of the present invention which include administering a combination of cetuximab and an ISC-4 prodrug in combination or separately, wherein the cancer is characterized by wild-type KRAS.

Methods of treating cancer in a subject in need thereof are provided according to aspects of the present invention which include administering a combination of cetuximab and an ISC-4 prodrug as a combination formulation or separately, wherein the cancer is characterized by wild-type KRAS such that the wild-type KRAS does not have an activating KRAS mutation.

Methods of treating cancer in a subject in need thereof are provided according to aspects of the present invention which include administering a combination of cetuximab and an ISC-4 prodrug as a combination formulation or separately, wherein the cancer is characterized by wild-type KRAS such that the wild-type KRAS does not have an activating KRAS mutation, in codon 12, 13 or 61, with reference to human KRAS.

Methods of treating cancer in a subject in need thereof are provided according to aspects of the present invention which include administering a combination of cetuximab and an ISC-4 prodrug as a combination formulation or separately, wherein the cancer is characterized by wild-type KRAS such that the wild-type KRAS does not have activating KRAS mutations Q61H, G12S, G12V, G12A or G13D, with reference to human KRAS.

Methods of treating colorectal cancer in a subject in need thereof are provided according to aspects of the present invention which include administering a combination of cetuximab and an ISC-4 prodrug in combination or separately, wherein the cancer is colorectal cancer characterized by wild-type KRAS.

Methods of treating colorectal cancer in a subject in need thereof are provided according to aspects of the present invention which include administering a combination of cetuximab and an ISC-4 prodrug as a combination formulation or separately, wherein the colorectal cancer is characterized by wild-type KRAS such that the wild-type KRAS does not have an activating KRAS mutation.

Methods of treating cancer in a subject in need thereof are provided according to aspects of the present invention which include administering a combination of cetuximab and an ISC-4 prodrug as a combination formulation or separately, wherein the cancer is colorectal cancer characterized by wild-type KRAS such that the wild-type KRAS does not have an activating KRAS mutation, in codon 12, 13 or 61, with reference to human KRAS.

Methods of treating cancer in a subject in need thereof are provided according to aspects of the present invention which include administering a combination of cetuximab and an ISC-4 prodrug as a combination formulation or separately, wherein the cancer is colorectal cancer characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include obtaining a first sample containing or suspected of containing cancer cells from the subject prior to administering the combination of cetuximab and an ISC-4 prodrug; administering a combination of cetuximab and an ISC-4 prodrug in combination or separately; obtaining a second sample containing or suspected of containing cancer cells from the subject after administering the combination of cetuximab and ISC-4 prodrug; and assaying the first and second samples for one or more markers of apoptosis, wherein increased apoptosis in the second sample compared to the first sample indicates therapeutic activity of the administered cetuximab and ISC-4 prodrug, thereby monitoring effectiveness of administering the combination of cetuximab and ISC-4 prodrug. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation, in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS. According to aspects of such methods, the cancer is colorectal cancer characterized by wild-type KRAS, such that the wild-type KRAS does not have an activating KRAS mutation. According to aspects of such methods, the cancer is colorectal cancer characterized by wild-type KRAS such that the wild-type KRAS does not have an activating KRAS mutation, in codon 12, 13 or 61, with reference to human KRAS. According to aspects of such methods, the cancer is colorectal cancer characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include obtaining a first sample containing or suspected of containing cancer cells from the subject prior to administering the combination of cetuximab and an ISC-4 prodrug; administering a combination of cetuximab and the ISC-4 prodrug in combination or separately; obtaining a second sample containing or suspected of containing cancer cells from the subject after administering the combination of cetuximab and the ISC-4 prodrug; and assaying the first and second samples for phospho-Akt, wherein decreased phospho-Akt in the second sample compared to the first sample indicates therapeutic activity of the administered cetuximab and the ISC-4 prodrug, thereby monitoring effectiveness of administering the combination of cetuximab and the ISC-4 prodrug. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation, in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS. According to aspects of such methods, the colorectal cancer is characterized by wild-type KRAS, such that the wild-type KRAS does not have an activating KRAS mutation. According to aspects of such methods, the colorectal cancer is characterized by wild-type KRAS such that the wild-type KRAS does not have an activating KRAS mutation, in codon 12, 13 or 61, with reference to human KRAS. According to aspects of such methods, the colorectal cancer is characterized by wild-type KRAS such that the wild-type KRAS does not have activating KRAS mutations Q61H, G12S, G12V, G12A or G13D, with reference to human KRAS.

According to aspects of methods of treating cancer of the present invention, the cetuximab and an ISC-4 prodrug are administered simultaneously or sequentially. In a non-limiting example, the cetuximab and the ISC-4 prodrug are administered sequentially within a period of time selected from: one hour, two hours, four hours, eight hours, twelve hours and twenty-four hours.

Methods of treating cancer in a subject in need thereof are provided according to aspects of the present invention which include administering a combination of cetuximab and an ISC-4 prodrug in combination or separately, wherein the ISC-4 prodrug is ISC-4 glucosinolate prodrug or a pharmaceutically acceptable salt thereof. The ISC-4 glucosinolate prodrug has the structural formula:

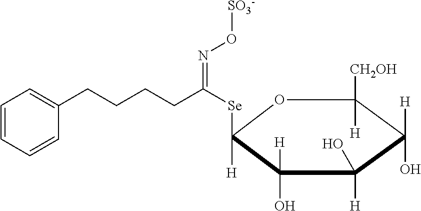

Methods of treating cancer in a subject in need thereof are provided according to aspects of the present invention which include administering a combination of cetuximab and an ISC-4 prodrug in combination or separately, wherein the cancer is characterized by wild-type KRAS and wherein the ISC-4 prodrug is ISC-4 glucosinolate prodrug or a pharmaceutically acceptable salt thereof. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include administering a combination of cetuximab and an ISC-4 prodrug in combination or separately, wherein the cancer is colorectal cancer characterized by wild-type KRAS and wherein the ISC-4 prodrug is ISC-4 glucosinolate prodrug or a pharmaceutically acceptable salt thereof. According to aspects of such methods, the colorectal cancer is characterized by wild-type KRAS, such that the wild-type KRAS does not have an activating KRAS mutation. According to aspects of such methods, the colorectal cancer is characterized by wild-type KRAS such that the wild-type KRAS does not have an activating KRAS mutation, in codon 12, 13 or 61, with reference to human KRAS. According to aspects of such methods, the colorectal cancer is characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include obtaining a first sample containing or suspected of containing cancer cells from the subject prior to administering the combination of cetuximab and an ISC-4 prodrug, wherein the ISC-4 prodrug is ISC-4 glucosinolate prodrug or a pharmaceutically acceptable salt thereof; administering a combination of cetuximab and the ISC-4 glucosinolate prodrug or a pharmaceutically acceptable salt thereof in combination or separately; obtaining a second sample containing or suspected of containing cancer cells from the subject after administering the combination of cetuximab and the ISC-4 glucosinolate prodrug or a pharmaceutically acceptable salt thereof; and assaying the first and second samples for one or more markers of apoptosis, thereby monitoring effectiveness of administering the combination of cetuximab and the ISC-4 glucosinolate prodrug or a pharmaceutically acceptable salt thereof. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation, in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS. According to aspects of such methods, the cancer is colorectal cancer characterized by wild-type KRAS, such that the wild-type KRAS does not have an activating KRAS mutation. According to aspects of such methods, the cancer is colorectal cancer characterized by wild-type KRAS such that the wild-type KRAS does not have an activating KRAS mutation, in codon 12, 13 or 61, with reference to human KRAS. According to aspects of such methods, the cancer is colorectal cancer characterized by wild-type KRAS such that the wild-type KRAS does not have activating KRAS mutations Q61H, G12S, G12V, G12A or G13D, with reference to human KRAS.

Methods of treating cancer in a subject in need thereof are provided according to aspects of the present invention which include obtaining a first sample containing or suspected of containing cancer cells from the subject prior to administering the combination of cetuximab and an ISC-4 prodrug, wherein the ISC-4 prodrug is ISC-4 glucosinolate prodrug or a pharmaceutically acceptable salt thereof; administering a combination of cetuximab and the ISC-4 glucosinolate prodrug or a pharmaceutically acceptable salt thereof in combination or separately; obtaining a second sample containing or suspected of containing cancer cells from the subject after administering the combination of cetuximab and the ISC-4 glucosinolate prodrug or a pharmaceutically acceptable salt thereof; and assaying the first and second samples for phospho-Akt, thereby monitoring effectiveness of administering the combination of cetuximab and the ISC-4 glucosinolate prodrug or a pharmaceutically acceptable salt thereof. According to aspects of such methods, the cancer is characterized by wild-type KRAS, such that the wild-type KRAS does not have an activating KRAS mutation. According to aspects of such methods, the cancer is characterized by wild-type KRAS such that the wild-type KRAS does not have an activating KRAS mutation, in codon 12, 13 or 61, with reference to human KRAS. According to aspects of such methods, the cancer is characterized by wild-type KRAS such that the wild-type KRAS does not have activating KRAS mutations Q61H, G12S, G12V, G12A or G13D, with reference to human KRAS. According to aspects of such methods, the cancer is colorectal cancer characterized by wild-type KRAS, such that the wild-type KRAS does not have an activating KRAS mutation. According to aspects of such methods, the cancer is colorectal cancer characterized by wild-type KRAS such that the wild-type KRAS does not have an activating KRAS mutation, in codon 12, 13 or 61, with reference to human KRAS. According to aspects of such methods, the cancer is colorectal cancer characterized by wild-type KRAS such that the wild-type KRAS does not have activating KRAS mutations Q61H, G12S, G12V, G12A or G13D, with reference to human KRAS.

According to aspects of methods of treating cancer of the present invention, the cetuximab and the ISC-4 glucosinolate prodrug or a pharmaceutically acceptable salt thereof, are administered simultaneously or sequentially.

In a non-limiting example, the cetuximab, and the ISC-4 glucosinolate prodrug or a pharmaceutically acceptable salt thereof, are administered sequentially within a period of time selected from: one hour, two hours, four hours, eight hours, twelve hours and twenty-four hours.

Pharmaceutical compositions according to aspects of the present invention include cetuximab, and the ISC-4 glucosinolate prodrug or a pharmaceutically acceptable salt thereof.

Commercial packages according to aspects of the present invention include cetuximab and the ISC-4 glucosinolate prodrug or a pharmaceutically acceptable salt thereof.

Commercial packages according to aspects of the present invention include a single pharmaceutical formulation including both cetuximab, and the ISC-4 glucosinolate prodrug or a pharmaceutically acceptable salt thereof.

Commercial packages according to aspects of the present invention include cetuximab and the ISC-4 glucosinolate prodrug or a pharmaceutically acceptable salt thereof, wherein the cetuximab is provided as a first pharmaceutical formulation and the ISC-4 glucosinolate prodrug or pharmaceutically acceptable salt thereof is provided as a separate second pharmaceutical formulation in the commercial package.

Compositions according to aspects of the present invention include the ISC-4 glucosinolate prodrug having the structural formula:

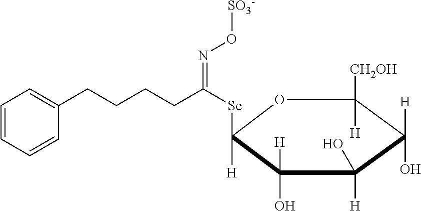

or a pharmaceutically acceptable salt thereof

Methods of assessing efficacy of treatment of cancer are provided according to aspects of the present invention which include: obtaining a first sample containing or suspected of containing cancer cells from a subject prior to administering cetuximab and ISC-4 together or separately; obtaining a second sample containing or suspected of containing cancer cells from the subject after administering the cetuximab and ISC-4; and assaying the first and second samples for one or more markers of apoptosis and/or assaying the first and second samples for phospho-Akt, wherein an increase in the one or more markers of apoptosis and a decrease in phosphor-Akt is indicative of therapeutic activity of administering both cetuximab and ISC-4 in combination, together or separately, thereby monitoring effectiveness of administering the combination of cetuximab and ISC-4.

Methods of assessing efficacy of treatment of cancer are provided according to aspects of the present invention which include: obtaining a first sample containing or suspected of containing cancer cells from a subject prior to administering cetuximab and an ISC-4 together or separately; obtaining a second sample containing or suspected of containing cancer cells from the subject after administering the cetuximab and the ISC-4 prodrug; and assaying the first and second samples for one or more markers of apoptosis and/or assaying the first and second samples for phospho-Akt, wherein an increase in the one or more markers of apoptosis and a decrease in phosphor-Akt is indicative of therapeutic activity of administering both cetuximab and the ISC-4 prodrug in combination, together or separately, thereby monitoring effectiveness of administering the combination of cetuximab and the ISC-4 prodrug.

Methods of assessing efficacy of treatment of cancer are provided according to aspects of the present invention which include: obtaining a first sample containing or suspected of containing cancer cells from a subject prior to administering cetuximab, and ISC-4 glucosinolate prodrug or pharmaceutically acceptable salt thereof, together or separately; obtaining a second sample containing or suspected of containing cancer cells from the subject after administering the cetuximab and the ISC-4 glucosinolate prodrug or pharmaceutically acceptable salt thereof; and assaying the first and second samples for one or more markers of apoptosis and/or assaying the first and second samples for phospho-Akt, wherein an increase in the one or more markers of apoptosis and a decrease in phosphor-Akt is indicative of therapeutic activity of administering both cetuximab and the ISC-4 glucosinolate prodrug or pharmaceutically acceptable salt thereof in combination, together or separately, thereby monitoring effectiveness of administering the combination of cetuximab and the ISC-4 glucosinolate prodrug or pharmaceutically acceptable salt thereof.

### BRIEF DESCRIPTION OF THE DRAWINGS

FIG. 1A is a graph showing results of cell viability assays and calculated EC50 values for indicated cell lines treated with ISC-4 or DMSO;

FIG. 1B is a graph showing the effect of ISC-4 treatment on cell cycle profiles of synchronous and asynchronous HCT116 cells;

FIG. 1C is a graph showing the effect of ISC-4 treatment on cell cycle profiles of synchronous and asynchronous HT-29 cells

FIG. 1D is a graph showing sub-G1 content of indicated colon cancer cell lines following ISC-4 treatment with 0, 1, 2, 4, 8, or 16 μM ISC-4;

FIG. 2 shows results of cell viability assays in SW480 and RKO colon cancer cell lines treated with ISC-4 (1, 2, or 4 μM) and indicated therapies at putative EC12.5, EC25, and EC50 alone and in combination;

FIG. 3A shows results of cell viability assays of human colon cancer cell line HT-29 treated with ISC-4 and cetuximab at indicated doses for 72 hours;

FIG. 3B shows results of cell viability assays of human colon cancer cell line RKO treated with ISC-4 and cetuximab at indicated doses for 72 hours;

FIG. 3C shows results of cell viability assays of human colon cancer cell line HCT116 treated with ISC-4 and cetuximab at indicated doses for 72 hours;

FIG. 3D shows results of cell viability assays of human colon cancer cell line DLD-1 treated with ISC-4 and cetuximab at indicated doses for 72 hours;

FIG. 3E is a graph showing results of a cell viability assay of wild-type and 5-FU-resistant RKO cells treated with 5-FU as indicated for 24 hours;

FIG. 3F is a graph showing results of 5-FU-resistant RKO cells treated with ISC-4 (2 μM) and cetuximab (1 μg/mL) for 24 hours;

FIG. 4A shows results of cell viability assays of RKO cells treated with ISC-4 (2 μM) and cetuximab (1 μg/mL) alone or in combination for the indicated time period;

FIG. 4B shows results of DAPI staining of RKO cells treated as in FIG. 4A for 12 hours;

FIG. 4C shows sub-G1 content of RKO cells treated with ISC-4 (2 μM) and cetuximab (1 μg/mL) alone or in combination for 12 hours;

FIG. 4D shows results of Caspase-Glo assay of RKO cells treated with ISC-4 (2 uM) in combination with cetuximab (0, 0.25, 0.5, or 1 μg/mL) at 24 hours post-treatment, top, and quantification of ISC-4 (2 μM) and cetuximab (1 μg/mL), bottom;

FIG. 5A shows results of Western blot analysis of RKO cells treated with ISC-4 (2 μM) and cetuximab (1 μg/mL) alone or in combination for 24 hours;

FIG. 5B shows results of Western blot analysis of RKO cells treated with ISC-4 (2 μM) and cetuximab (1 μg/mL) alone or in combination for indicated time periods;

FIG. 5C shows results of Western blot analysis of indicated human colon cancer cell lines following treatment with the combination (Rx) of ISC-4 (2 μM) and cetuximab (1 μg/mL) for 8 hrs, *P<0.05 compared to control;

FIG. 6A is a graph showing relative tumor sizes of 5-FU-resistant RKO xenografts at 4 days post-treatment with a single dose of ISC-4 (3 mg/kg, i.p.), cetuximab (10 mg/kg, i.v.), or the combination (“combo”);

FIG. 6B shows results of hematoxylin and eosin (H&E) staining and TUNEL staining of xenograft tumors harvested 24 hours after treatment;

FIG. 6C shows results of treatment of athymic female nude mice harboring established HT-29 xenograft tumors with ISC-4 (3 mg/kg, i.v.), cetuximab (10 mg/kg, i.v.), the combination, or cetuximab and 5-FU (25 mg/kg, i.v.) once per week starting on day 0;

FIG. 7A shows phase-contrast images of RKO cells treated with ISC-4 (2 μM) and cetuximab (1 μg/mL) alone or in combination for 12 hours;

FIG. 7B is a graph showing results of flow cytometry analysis of Ki-67 expression in RKO cells treated with ISC-4 (2 μM) and cetuximab (1 μg/mL) alone or in combination;

FIG. 7C shows Western bot analysis of Ki-67 expression in RKO cells treated with ISC-4 (2 μM) and cetuximab (1 μg/mL) alone or in combination;

FIG. 7D shows results of Western blot analysis of RKO cells treated with ISC-4 (2 μM) and cetuximab (1 μg/mL) alone or in combination for 24 hours;

FIG. 8A is a graph showing change in body weight of mice receiving ISC-4 (3 mg/kg, i.p.), cetuximab (10 mg/kg, i.v.), or the combination (n≧5) twice a week for 2 weeks;

FIG. 8B shows results of H&E staining of liver tissue harvested from mice at 24 hours post-treatment with ISC-4 (3 mg/kg, i.p.), cetuximab (10 mg/kg, i.v.), or the combination;

FIG. 8C is a graph showing terminal tumor volume and tumor weight for HT-29 xenograft described in FIG. 6C; and

FIG. 8D is a graph showing mouse body weight at endpoint, which was three days following the last dose (n≧8), error bars indicate SEM of replicates.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 9780470151808.

The singular terms “a,” “an,” and “the” are not intended to be limiting and include plural referents unless explicitly stated otherwise or the context clearly indicates otherwise.

Synergistic effects of combination treatment including administration of ISC-4 and cetuximab is unexpectedly found as described herein.

Methods are provided according to the present invention for treating cancer in a subject in need thereof which include administering a combination of cetuximab and ISC-4 as a combination formulation or separately, wherein administration of the combination provides a synergistic effect.

The term “ISC-4” refers to the compound having the structural formula:

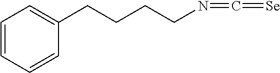 [SMILES: [Se]=C=NCCCCc1ccccc1]

The compound ISC-4 can be synthesized using standard chemical synthetic methodology, for example as described in Sharma, A. K., et al., J. of Med. Chem., 2008, 51(24):7820-7826.

Cancers treated using methods and compositions described herein are characterized by abnormal cell proliferation including, but not limited to, pre-neoplastic hyperproliferation, cancer in-situ, neoplasms and metastasis.

Methods of treatment of a subject having, or at risk of having cancer characterized by wild-type KRAS are provided according to aspects of the present invention which include administering a combination of cetuximab and ISC-4 as a combination formulation or separately, wherein administration of the combination provides a synergistic effect.

KRAS, also called GTPase KRas and V-Ki-ras2 Kirsten rat sarcoma viral oncogene homolog, is well known in the art, along with mutations of KRAS associated with overactivated KRAS and cancer, see S. M. Anderson, Expert Review of Molecular Diagnostics, 2011, 11(6):635-642; Schimanski et al., Cancer Res, 1999, 59:5169-5175; Chang et al., BMC Cancer 9:179, 2009; and Jan\check{c}ik et al., Clinical Relevance of KRAS in Human Cancers, Journal of Biomedicine and Biotechnology, 2010, Article ID 150960, Epub Jun. 7, 2010.

Activating KRAS mutations are well-known and include, but are not limited to, those in codons 12 and 13, as well as in codon 61, with reference to human KRAS. Examples of well-known activating KRAS mutations include, without limitation, Q61H, G12S, G12V, G12A and G13D, with reference to human KRAS. These and other well-known activating KRAS mutations are described in S. M. Anderson, Expert Review of Molecular Diagnostics, 2011, 11(6):635-642; Schimanski et al., Cancer Res, 1999, 59:5169-5175; Chang et al., BMC Cancer 9:179, 2009; and Jan\check{c}ik et al., Clinical Relevance of KRAS in Human Cancers, Journal of Biomedicine and Biotechnology, 2010, Article ID 150960, Epub Jun. 7, 2010.

The mutation status of KRAS can be assayed in a test sample obtained from a subject.

A test sample can be any biological fluid, cell or tissue of a subject that includes or is suspected of including cancer cells or circulating DNA derived from cancer cells, illustratively including blood, plasma, serum, urine, saliva, ascites, cerebrospinal fluid, cerebroventricular fluid, pleural fluids, pulmonary and bronchial lavage samples, mucous, sweat, tears, semen, bladder wash samples, amniotic fluid, lymph, peritoneal fluid, synovial fluid, bone marrow aspirate, tumor cells or tissue, organ cells or tissue, such as biopsy material.

The mutation status of KRAS can be assayed by any of various methodologies including, but not limited to, protein or peptide sequencing, nucleic acid assay and immunoassay. Exemplary methods for determining the mutation status of KRAS are described in S. M. Anderson, Expert Review of Molecular Diagnostics, 2011, 11(6):635-642; Schimanski et al., Cancer Res, 1999, 59:5169-5175; Chang et al., BMC Cancer 9:179, 2009; and Jan\check{c}ik et al., Clinical Relevance of KRAS in Human Cancers, Journal of Biomedicine and Biotechnology, 2010, Article ID 150960, Epub Jun. 7, 2010.

Assays for detecting KRAS nucleic acids, particularly mRNA or cDNA, include, but are not limited to, sequencing; polymerase chain reactions (PCR) such as RT-PCR; dot blot; in situ hybridization; Northern blot; and RNase protection.

Immunoassay methods can be used to assay KRAS mutation status in a sample, including, but not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay.

Methods of treatment of a subject having, or at risk of having cancer characterized by resistance to 5-fluorouracil are provided according to aspects of the present invention which include administering a combination of cetuximab and ISC-4 as a combination formulation or separately, wherein administration of the combination provides a synergistic effect.

Methods of treatment of a subject having, or at risk of having colorectal cancer characterized by wild-type KRAS are provided according to aspects of the present invention which include administering a combination of cetuximab and ISC-4 as a combination formulation or separately, wherein administration of the combination provides a synergistic effect.

Methods and compositions of the present invention can be used for prophylaxis as well as amelioration of signs and/or symptoms of cancer. The terms “treating” and “treatment” used to refer to treatment of a cancer in a subject include: preventing, inhibiting or ameliorating the cancer in the subject, such as slowing progression of the cancer and/or reducing or ameliorating a sign or symptom of the cancer.

A therapeutically effective amount of cetuximab and ISC-4 administered as a combination treatment of the present invention is an amount which has a beneficial effect in a subject being treated. In subjects having cancer or at risk for having cancer, such as a condition characterized by abnormal cell proliferation including, but not limited to, pre-neoplastic hyperproliferation, cancer in-situ, neoplasms, metastasis, a tumor, a benign growth or other condition responsive to a composition of the present invention, a therapeutically effective amount of a composition of the present invention is effective to ameliorate or prevent one or more signs and/or symptoms of the condition.

A therapeutically effective amount of cetuximab and ISC-4 administered as a combination treatment of the present invention is effective to detectably increase apoptosis and/or decrease proliferation of cells of a cancer. A therapeutically effective amount of cetuximab and ISC-4 administered as a combination treatment of the present invention is effective to detectably decrease phospho-Akt in cells of a cancer.

A subject treated according to methods and using compositions of the present invention can be mammalian or non-mammalian. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. A non-mammalian subject can be any non-mammal including, but not limited to, a bird such as a duck, goose, chicken, or turkey. Subjects can be either gender and can be any age. In aspects of methods including administration of an inventive pharmaceutical composition to a subject, the subject is human. The terms “subject” and “patient” are used interchangeably herein.

Combinations of cetuximab, ISC-4 and one or more additional therapeutic agents are administered according to aspects of the present invention.

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present invention include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal antiinflammatory agents, steroids and vasoactive agents.

Combination therapies including administration of ISC-4 and cetuximab show synergistic effects.

According to aspects of the present invention, combination therapies include: (1) pharmaceutical compositions that include a pharmaceutical combination composition including ISC-4 and cetuximab formulated together in a single pharmaceutical composition; and/or (2) co-administration of ISC-4 and cetuximab wherein ISC-4 and cetuximab have not been formulated in the same composition. When using separate formulations ISC-4 may be administered at the same time, intermittent times, staggered times, prior to, subsequent to, or combinations thereof, with reference to cetuximab.

According to aspects of the present invention, combination therapies include: (1) pharmaceutical compositions that include a pharmaceutical combination composition including ISC-4 and cetuximab formulated together with one or more additional therapeutic agents in a single pharmaceutical composition; (2) co-administration of ISC-4, cetuximab and one or more additional pharmaceutical agents wherein ISC-4, cetuximab and the one or more additional pharmaceutical agents have not been formulated in the same composition; and/or (3) co-administration of ISC-4, cetuximab and one or more additional pharmaceutical agents wherein two or more, but not all, of: ISC-4, cetuximab and the one or more additional pharmaceutical agents are formulated in the same composition. When using separate formulations each of ISC-4, cetuximab and one or more additional pharmaceutical agents may be administered at the same time, intermittent times, staggered times, prior to, subsequent to, or combinations thereof, with reference to each of the other components.

Combination treatments can allow for reduced effective dosage and increased therapeutic index of the pharmaceutical composition including ISC-4 and cetuximab.

An additional pharmaceutical agent is an anti-cancer agent according to aspects of the present invention.

Anti-cancer agents are described, for example, in Goodman et al., Goodman and Gilman's The Pharmacological Basis of Therapeutics, 8th Ed., Macmillan Publishing Co., 1990.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bevacizumab, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

According to aspects of the present invention, one or more correlative biomarkers of therapeutic activity of cetuximab and ISC-4 administered as a combination treatment of the present invention to treat cancer in a subject in need thereof are assayed to assess treatment of the cancer in the subject. Thus, for example, the level of phospho-Akt is a correlative biomarker of therapeutic activity of cetuximab and ISC-4 administered as a combination treatment of the present invention to treat cancer in a subject in need thereof and a decrease in phospho-Akt in cancer cells is indicative of efficacy of cetuximab and ISC-4 administered as a combination treatment of the present invention to treat cancer in a subject in need thereof. Levels of phospho-Akt are measured according to standard methodologies, for example as described herein. Biomarkers of apoptosis are correlative biomarkers of therapeutic activity of cetuximab and ISC-4 administered as a combination treatment of the present invention to treat cancer in a subject in need thereof and an increase in one or more biomarkers of apoptosis in cancer cells is indicative of efficacy of cetuximab and ISC-4 administered as a combination treatment of the present invention to treat cancer in a subject in need thereof. Biomarkers of apoptosis include, but are not limited to, detection of DNA fragmentation, characteristic morphological changes distinct from necrosis and activation of caspase-3. Biomarkers of apoptosis are measured according to standard methodologies, for example as described herein.

According to aspects of the present invention, assays for effects of combination treatment with cetuximab and ISC-4 are used to monitor a subject. Thus, for example, a test sample is obtained from the subject before treatment according to a method of the present invention and at one or more times during and/or following treatment in order to assess effectiveness of the treatment. In a further example, a test sample is obtained from the subject at various times in order to assess the course or progress of disease or healing.

In particular aspects, one or more additional biomarkers are assayed in a test sample obtained from a subject to aid in monitoring treatment with a pharmaceutical composition of the present invention. For example, one or more of phospho-Akt and/or detection of apoptosis of cancer cells is assayed in a test sample obtained from a subject to aid in monitoring treatment with a pharmaceutical composition of the present invention.

Optionally, a method of treating cancer in a subject in need thereof further includes an adjunct anti-cancer treatment. An adjunct anti-cancer treatment can be a radiation treatment of a subject or an affected area of a subject's body.

The dosage of cetuximab, ISC-4 and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

In general it is contemplated that a daily dosage of cetuximab, ISC-4 and any optional additional therapeutic agent is in the range of about 0.001 to 100 milligrams per kilogram of a subject's body weight. A daily dose may be administered as two or more divided doses to obtain the desired effect. A pharmaceutical composition including any one or more of: cetuximab, ISC-4 and any optional additional therapeutic agent, may also be formulated for sustained release to obtain desired results.

In particular aspects of inventive methods, the amount of the adjunct anti-cancer treatment and/or anti-cancer agent administered is less than an amount of the adjunct anti-cancer treatment and/or anti-cancer agent necessary to achieve a therapeutic effect if administered without a combination treatment of the present invention including administration of ISC-4 and cetuximab. Thus, in particular aspects of the present invention, the amount of an anti-cancer treatment and/or agent administered is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the adjunct anti-cancer treatment and/or agent necessary to achieve a therapeutic effect when administered without a combination treatment of the present invention including administration of ISC-4 and cetuximab.

Methods of the present invention include administration of a pharmaceutical composition of the present invention by a route of administration including, but not limited to, oral, rectal, nasal, pulmonary, epidural, ocular, otic, intraarterial, intracardiac, intracerebroventricular, intradermal, intravenous, intramuscular, intraperitoneal, intraosseous, intrathecal, intravesical, subcutaneous, topical, transdermal, and transmucosal, such as by sublingual, buccal, vaginal, and inhalational, routes of administration.

Prodrugs

One or more prodrugs of ISC-4 is administered in combination with cetuximab according to aspects of the present invention to achieve the benefits of ISC-4 administration in combination with cetuximab. An ISC-4 prodrug is optionally administered in combination with ISC-4 and cetuximab. An ISC-4 prodrug substitutes for ISC-4 in methods of treatment or compositions described herein or may be used in addition to ISC-4 in methods of treatment or compositions described herein.

An ISC-4 prodrug is a form of ISC-4 covalently bound to a moiety, or moieties, released from the ISC-4 prodrug yielding ISC-4. Examples of prodrug forms are described in Sloan, K. B., Prodrugs, M. Dekker, New York, 1992; and Testa, B. and Mayer, J. M., Hydrolysis in drug and prodrug metabolism: chemistry, biochemistry, and enzymology, Wiley-VCH, Zurich, 2003.

A particular ISC-4 prodrug is a glucosinolate prodrug of ISC-4. The ISC-4 glucosinolate prodrug will be synthesized as outlined in the scheme below. This glucosinolate prodrug of ISC-4, upon interaction with myrosinase enzyme in vitro or in vivo, would release the active ISC-4. The glucosinolate prodrug of ISC-4 is expected to be water soluble.

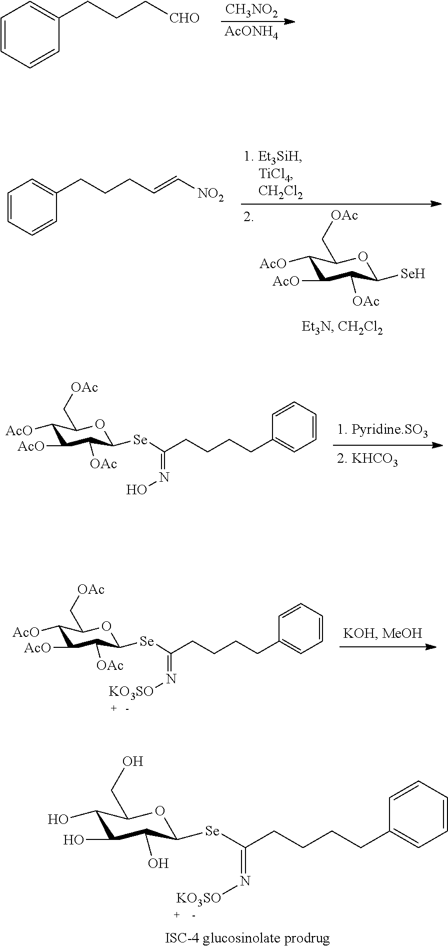

ISC-4 glucosinolate prodrug (I) is administered in combination with cetuximab according to aspects of the present invention to achieve the benefits of ISC-4 administration in combination with cetuximab. ISC-4 glucosinolate prodrug (I) is optionally administered in combination with ISC-4 and cetuximab.

ISC-4 glucosinolate prodrug according to (I) is optionally provided as a pharmaceutically acceptable salt.

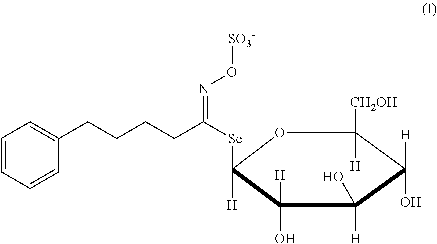

A pharmaceutically acceptable salt formulation of the ISC-4 prodrug of structure (I) can be any salt form of the ISC-4 prodrug of structure (I) that is generally non-toxic to an intended recipient and which does not significantly inhibit activity of the ISC-4 prodrug of structure (I) or other active agent included in the composition. For example, a potassium salt form of the ISC-4 prodrug of structure (I) is shown in the synthetic scheme above.

Combination Pharmaceutical Compositions

A combination pharmaceutical composition including both ISC-4 and cetuximab according to the invention generally includes about 0.1-99% of ISC-4, about 0.1-99% of cetuximab; and a pharmaceutically acceptable carrier.

A combination pharmaceutical composition including cetuximab and ISC-4 and/or a prodrug of ISC-4 according to the invention generally includes about 0.1-99% of ISC-4 and or a prodrug of ISC-4, about 0.1-99% of cetuximab; and a pharmaceutically acceptable carrier.

A combination pharmaceutical composition including cetuximab and ISC-4 and/or ISC-4 glucosinolate prodrug (I) according to the invention generally includes about 0.1-99% of ISC-4 and/or ISC-4 glucosinolate prodrug (I), about 0.1-99% of cetuximab; and a pharmaceutically acceptable carrier.

A pharmaceutical composition of the present invention may be in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. Liposomes and emulsions are well-known types of pharmaceutical formulations that can be used to deliver a pharmaceutical agent, particularly a hydrophobic pharmaceutical agent. Pharmaceutical compositions of the present invention generally include a pharmaceutically acceptable carrier such as an excipient, diluent and/or vehicle. Delayed release formulations of compositions and delayed release systems, such as semipermeable matrices of solid hydrophobic polymers can be used.

A pharmaceutical formulation of a composition of the present invention can include a pharmaceutically acceptable carrier. The term “pharmaceutically acceptable carrier” refers to a carrier which is suitable for use in a subject without undue toxicity or irritation to the subject and which is compatible with other ingredients included in a pharmaceutical composition.

Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alignates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present invention may include a colorant, a stabilizer, a wetting agent, an emulsifying agent, a suspending agent, a sweetener, a flavoring, or a perfuming agent.

For example, a composition for parenteral administration may be formulated as an injectable liquid. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavoring, or a perfuming agent.

Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Ionotophoresis and/or sonophoresis can be used to enhance transdermal delivery.

Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount.

Opthalmic formulations of one or more active agents can include ingredients such as a preservative, a buffer and a thickening agent.

Suitable surface-active agents useful as a pharmaceutically acceptable carrier or excipient in the pharmaceutical compositions of the present invention include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphoshatidyl-choline and their mixtures.

Suitable non-ionic surfactants useful as pharmaceutically acceptable carriers or excipients in the pharmaceutical compositions of the present invention include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present invention include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further sub-stituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

In particular aspects, a pharmaceutically acceptable carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate pharmaceutically acceptable carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Detailed information concerning customary ingredients, equipment and processes for preparing dosage forms is found in Pharmaceutical Dosage Forms: Tablets, eds. H. A.

Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

Commercial packages according to aspects of the present invention include cetuximab and ISC-4, formulated in combination or separately. Instructions for administering the cetuximab and ISC-4 are included according to aspects of the invention. One or more ancillary components is optionally included in commercial packages of the present invention, such as a buffer or diluent.

Aspects of inventive compositions and methods are illustrated in the following examples. These examples are provided for illustrative purposes and are not considered limitations on the scope of inventive compositions and methods.

### EXAMPLES

### Cell Culture, Cell Viability Assays, and Reagents

Cell lines are obtained from ATCC and cultured in ATCC-recommended media in a humidified incubator at 5% CO₂ and 37° C. For cell viability assays, cells are seeded into 96-well black-walled plates at a concentration of 1×10⁵ cells per mL in fresh media and in a volume of 100 μL per well. Cells are allowed to adhere overnight and are treated the next day as indicated. At endpoint, CellTiter-Glo (Promega) assays are performed according to the manufacturer's protocol, and the bioluminescent readout is recorded on an IVIS imaging system (Xenogen). For cell synchronization, cells are incubated with 200 ng/mL nocodazole for 16 hours prior to treatment. Chloroquine is obtained from Sigma. zVAD-fmk is obtained from Promega and used at a working concentration of 25 μM. ISC-4 is synthesized as described in Sharma, A. K., et al., J. of Med. Chem., 2008, 51(24):7820-7826.

Flow Cytometry

For sub-G1 DNA content analysis, cells are trypsinized at the indicated time points and fixed in 80% ethanol at 4° C. for a minimum of 30 minutes. Fixed cells are then stained with propidium iodide in the presence of RNase and analyzed on an Epics Elite flow cytometer (Beckman Coulter). For Ki-67 expression, cells are ethanol fixed, as described above, and immunostained with an anti-Ki-67 antibody (Sigma) at 1:500 for 30 minutes. Cells are subsequently incubated with Alexafluor 488-conjugated antibody at 1:500 in PBS for 30 minutes and resuspended in PBS for analysis.

Western Blot Analysis

Cells are treated in log-phase growth, harvested by cell scraping, centrifuged, and lysed on ice for 2 hours with cell-lysis buffer. The supernatant is collected following centrifugation, and protein concentration is determined using the Bio-Rad protein assay (Bio-Rad Laboratories). Samples are electrophoresed under reducing conditions on NuPAGE 4-12% Bis-Tris gels (Invitrogen), transferred to PVDF, and blocked in 10% non-fat milk in TBST for 1 hour. Membranes are then incubated with primary antibodies obtained from Cell Signaling at 1:1000 in 2% non-fat milk in TBST overnight at 4° C. Membranes are washed in TBST, incubated with the appropriate HRP-conjugated secondary antibody (Thermo-Scientific) for 1 hour, washed in TBST, and visualized using ECL-Plus (Amersham) and X-Ray film (Thermo-Scientific).

In Vivo Studies

Athymic female nude mice (Charles River Laboratories) are inoculated with 1×10⁶ of 5-FU-resistant RKO or HT-29 cells in each rear flank as a 200 μL suspension of 1:1 Matrigel (BD):PBS. Treatment is initiated once tumors reached a mean volume of 1650 mm³, intraperitoneal or intravenous injections are given at a total volume of 200 μL in DMSO. For tissue analysis, tissue is harvested from euthanized mice and fixed in 4% paraformaldehyde in PBS for 48 hours. Tissue is paraffin-embedded and sectioned. H&E staining (Daiko) and TUNEL staining (Millipore) are carried out according to the manufacturer's protocols. For serum chemistry assays, 1 mL of blood is harvested from anesthetized mice by terminal cardiac puncture of the left ventricle. For serum chemistry, 500 μL is placed into a microfuge tube and allowed to clot for 30 minutes at room temperature followed by centrifugation. Serum is removed, centrifuged again to remove any additional debris prior to analysis.

Statistics

Pairwise comparisons are assessed by the Student's two-tailed t-test in Microsoft Excel. Combination indices are computed with CalcuSyn software (BioSoft) using the Chou-Talalay method described in Chou, T.-C., Pharmacological Reviews, 2006, 58(3):621-681.

Defining the ISC-4 In Vitro Activity Profile

The in vitro activity of ISC-4 is tested in a panel of human cancer cell lines to characterize its spectrum of activity.

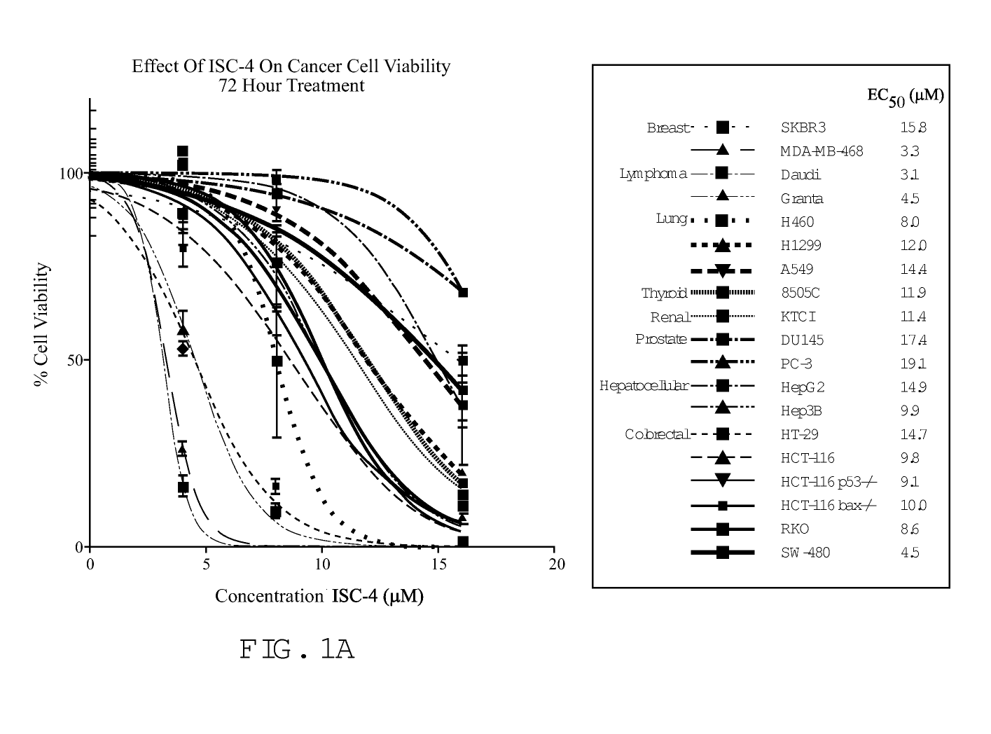

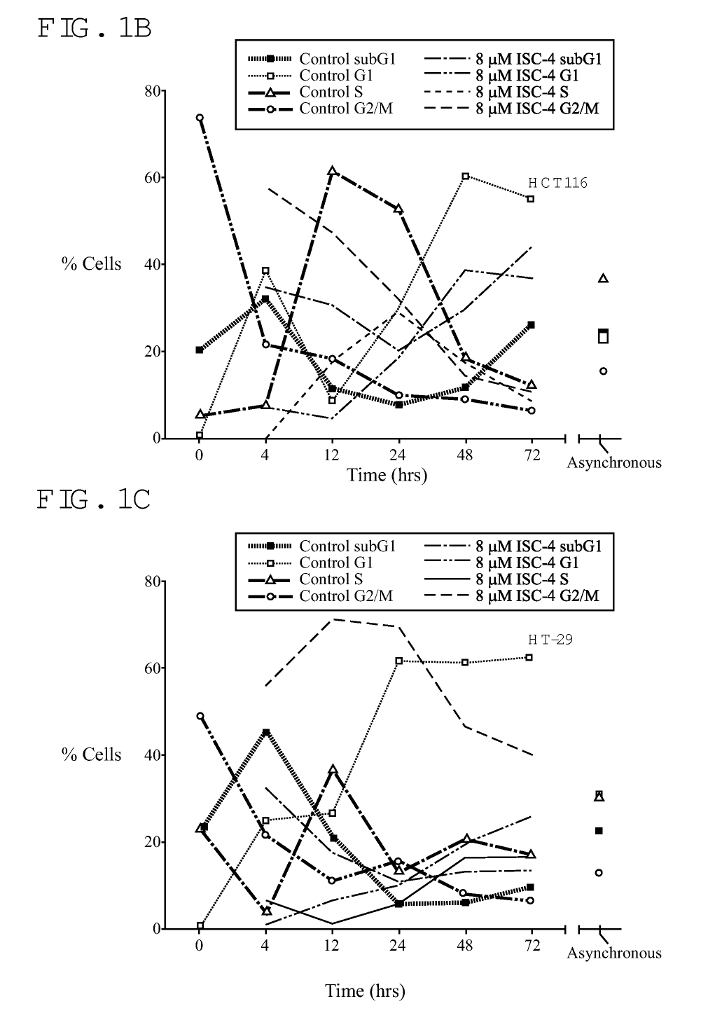

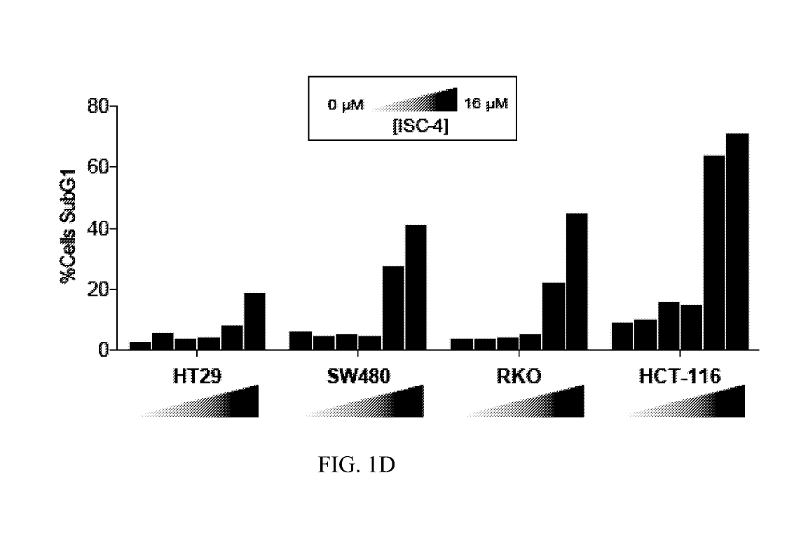

FIG. 1A shows results of cell viability assays and calculated EC₅₀ values for indicated cell lines treated with ISC-4 or DMSO (72 hr, n=3). Among the tested cell lines, the human lymphoma cell lines Daudi and Granta are the most sensitive, and the human prostate cancer cell lines PC3 and DU145 are the least sensitive in terms of EC₅₀ values as shown in FIG. 1A. With the exception of HT-29, human colon cancer cell lines are moderately sensitive to ISC-4 treatment. The isogenic HCT116 cell lines indicate that ISC-4 activity is likely p53- and Bax-independent.

The effect of ISC-4 treatment on cell cycle profiles of synchronous and asynchronous HCT116 (FIG. 1B) and HT-29 (FIG. 1C) cell lines is shown. Cell cycle analysis of ISC-4-treated synchronized HCT116 and HT-29 human colon cancer cell lines reveal a modest increase in sub-G1 content and a decrease in the rate of cell cycle progression as shown in FIG. 1B. ISC-4-induced sub-G1 content is dose-dependent and generally becomes apparent at >8 μM as shown in FIG. 1C. Thus, ISC-4 has modest single agent activity against human colon cancer cells by causing cell death and a decrease in proliferation. FIG. 1D shows sub-G1 content of indicated colon cancer cell lines following ISC-4 treatment with 0, 1, 2, 4, 8, or 16 μM ISC-4.

Identification of Synergistic Drug Combinations

The SW480 and RKO human colon cancer cell lines are used for initial profiling based on their heterogeneous oncogenic genetic alterations. SW480 has mutant p.53, mutant KRAS, and wild-type BRAF whereas RKO has wild-type p53, wild-type KRAS, and mutant BRAF genes, see Ikediobi, O. N., et al., Molecular Cancer Therapeutics, 2006, 5(11):2606-2612. FIG. 2 shows results of cell viability assays in SW480 and RKO colon cancer cell lines treated with ISC-4 (1, 2, or 4 μM) and indicated therapies at putative EC_{12.5}, EC₂₅, and EC₅₀ alone and in combination (n=3). Doses used are indicated in Table I. Among the test panel of chemotherapies and targeted agents, combinatorial activity is observed in at least one cell line when ISC-4 is combined with sorafenib, gefitinib, gemcitabine, cisplatin, bortezomib, imatinib, or cetuximab, as shown in FIG. 2, Table I and Table II.

Table I: Doses selected for approved antitumor agents in combination with ISC-4. EC_{12.5}, EC₂₅, and EC₅₀ values are estimated from the literature and doses are employed in experiments for which the results are shown in FIG. 2.

TABLE I

| Therapy | EC_{12.5} | | EC₂₅ | | EC₅₀ | |
|---|---|---|---|---|---|---|
| Lapatinib | 1.75 | uM | 3.5 | uM | 7 | uM |
| Cisplatin | 1.125 | uM | 2.25 | uM | 4.5 | uM |
| Panitumumab | 0.75 | ug/mL | 1.5 | ug/mL | 3 | ug/mL |
| Cetuximab | 0.25 | ug/mL | 0.5 | ug/mL | 1 | ug/mL |
| Sorafenib | 8 | uM | 16 | uM | 32 | uM |
| Trastuzumab | 62.5 | ng/mL | 125 | ng/mL | 250 | ng/mL |
| Gefitinib | 5.75 | uM | 11.5 | uM | 23 | uM |
| Imatinib | 1.25 | uM | 2.5 | uM | 5 | uM |
| Vincristine | .035 | nM | .07 | nM | .14 | nM |
| Pemetrexed | 0.25 | ug/mL | 0.5 | ug/mL | 1 | ug/mL |
| Doxorubicin | 16.25 | uM | 32.5 | uM | 65 | uM |
| Cladribine | 25 | nM | 50 | nM | 100 | nM |
| Docetaxel | 2.5 | uM | 5 | uM | 10 | uM |
| Paclitaxel | 2.5 | nM | 5 | nM | 10 | nM |
| Etoposide | 1.25 | uM | 2.5 | uM | 5 | uM |
| Oxaliplatin | 75 | nM | 150 | nM | 300 | nM |
| Irinotecan | 3.38 | uM | 6.75 | uM | 13 | uM |
| 5-FU | 4.5 | uM | 9 | uM | 18 | uM |
| Gemcitabine | 0.5 | uM | 1 | uM | 2 | uM |

Table II shows a summary of combinatorial effects of ISC-4 with approved antitumor agents. Combinatorial activities of ISC-4 and each listed drug are compared to monoagent activities of each drug alone by cell viability assays and determined to be uncooperative (−), cooperative (+), synergistic (*), or ambiguous (?). Drug combinations exhibiting cooperative activity with ISC-4 in at least one cell line are sorafenib, genfitinib, gemcitabine, cisplatin, bortezomib and imatinib whereas the combination of cetuximab and ISC-4 shows synergy.

TABLE II

| Class | Therapy | SW480 | RKO |
|---|---|---|---|
| Small | Sorafenib | + | + |
| molecules | Gefitinib | + | + |
| | Gemcitabine | − | + |
| | Cisplatin | + | − |
| | Bortezomib | ? | + |
| | Imatinib | − | + |
| | Vincristine | − | − |
| | Pemetrexed | − | − |
| | Doxorubicin | − | ? |
| | Cladribine | − | − |
| | Docetaxel | − | − |
| | Paclitaxel | − | − |
| | Etoposide | − | − |
| | Oxaliplatin | − | − |
| | Irinotecan | − | − |
| | 5-FU | − | − |
| Antibodies | Lapatinib | − | − |
| | Cetuximab | − | * |
| | Panitumumab | − | − |
| | Trastuzumab | − | − |

The combination of ISC-4 and cetuximab is the only synergistic combinatorial therapy observed under the tested conditions. Furthermore, this synergy is observed in the RKO cell line, which harbors wild-type KRAS, and not in the SW480 cell line that harbors KRAS^{G12V}. This observation is in accordance with the requirement of wild-type KRAS for the clinical efficacy of cetuximab in colon cancer as described in Lievre, A., et al., Cancer Res, 2006, 66(8):3992-3995; and Karapetis, C. S., et al., New England Journal of Medicine, 2008, 359(17):1757-1765.

ISC-4 and Cetuximab Synergistically Inhibit Wild-Type KRAS Tumor Cell Proliferation

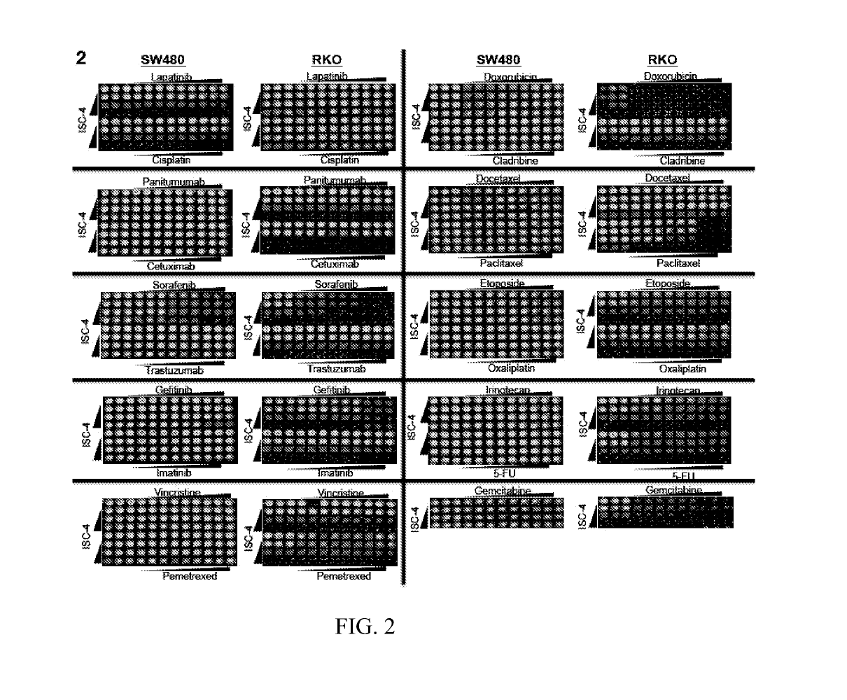

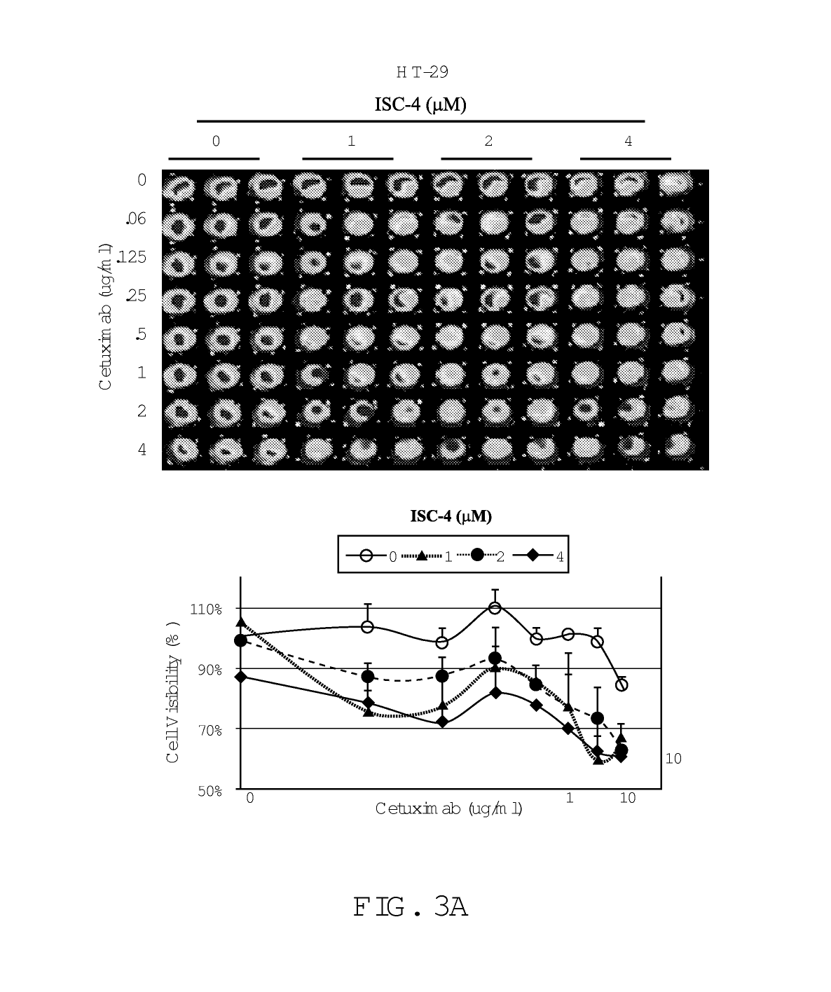

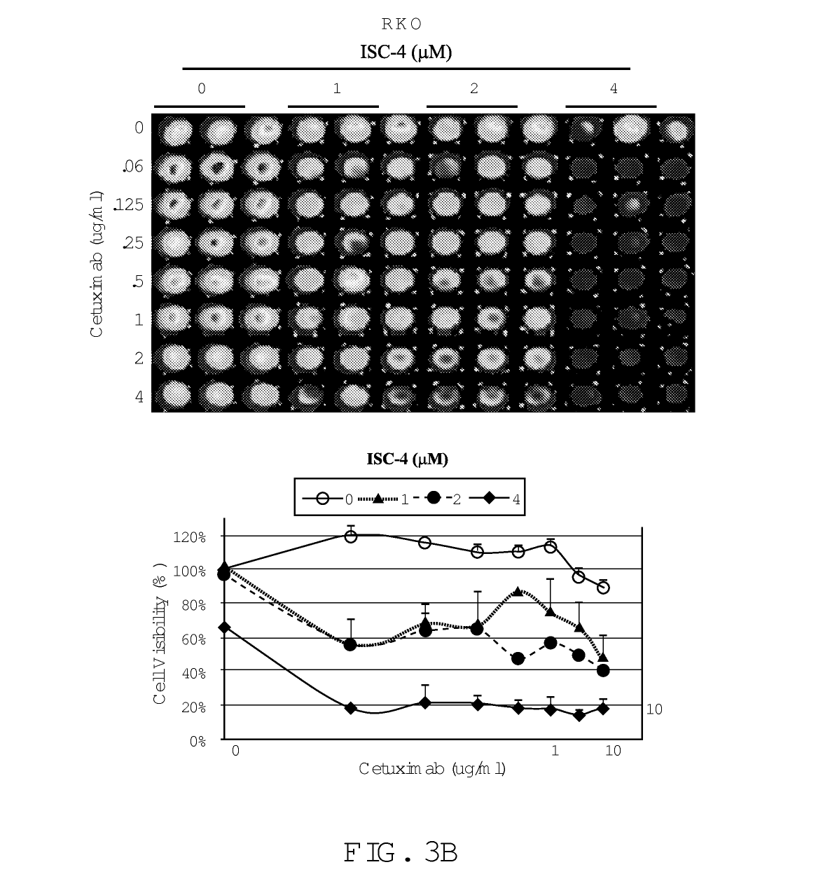

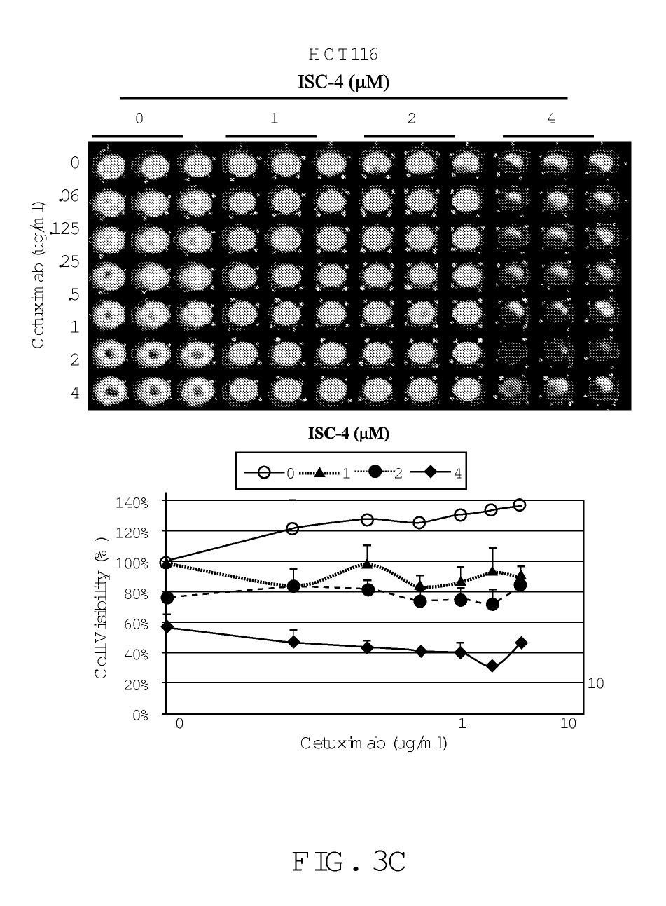

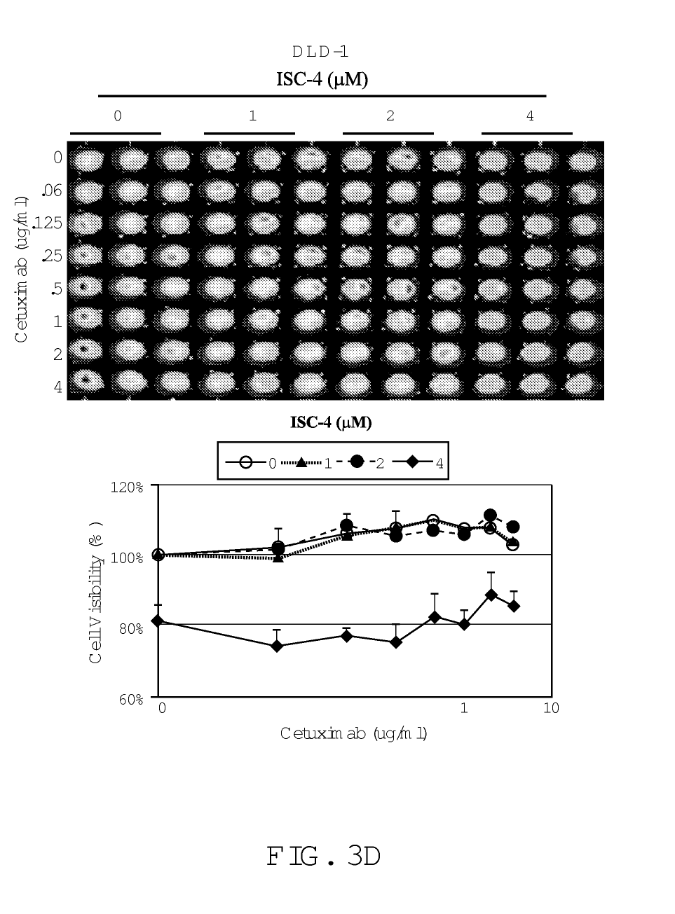

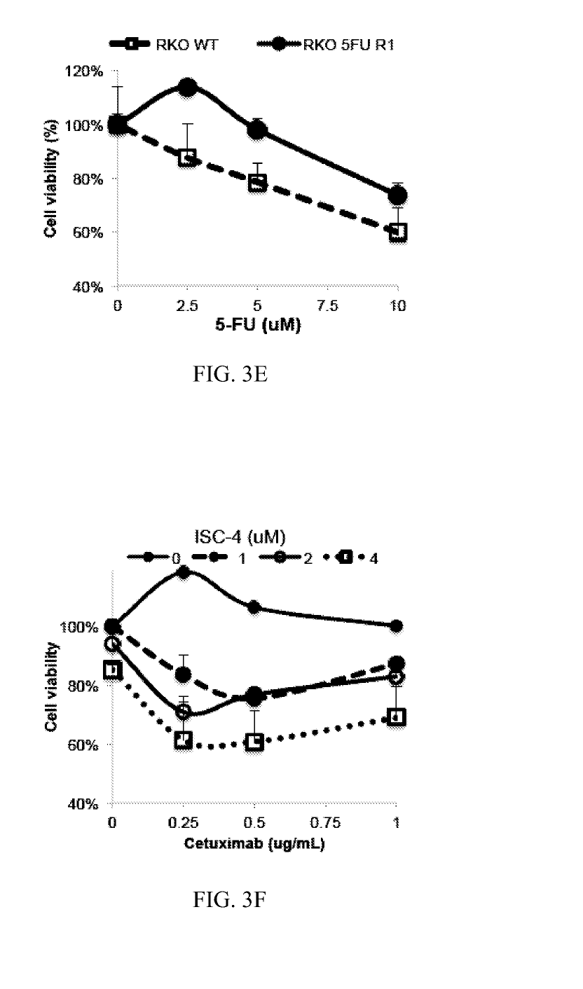

The synergistic activity of ISC-4 and cetuximab is evaluated in several human colon cancer cell lines in this example. FIGS. 3A-3D, 3E and 3F show that ISC-4 and cetuximab synergize in human colon cancer cells with wild-type KRAS genes independently of 5-FU sensitivity. FIGS. 3A-3D show results of cell viability assays of human colon cancer cell lines treated with ISC-4 and cetuximab at indicated doses for 72 hours (n=3).

Synergistic activity is observed in HT-29 and RKO cell lines, which have wild-type KRAS genes, and not in HCT116, DLD-1, and other colon cancer cell lines with mutant KRAS genes as shown in FIGS. 3A-3D; and Table III.

Table III: Combination indices for the ISC-4 and cetuximab in wild-type KRAS human colon cancer cell lines. Combinatorial activity in RKO and HT-29 cell lines quantified and shown in FIGS. 3A-3D are assessed by the Chou-Talalay method and results shown in Table III.

TABLE III

| | | | 0.06 | 0.125 | 0.25 | 0.5 | 1 | 2 | 4 |
|---|---|---|---|---|---|---|---|---|---|
| | | | Cetuximab (ug/mL) | | | | | | |
| HT-29 | ISC-4 | 1 | 0.245 | 0.251 | 0.312 | 0.298 | 0.268 | 0.234 | 0.29 |
| | (uM) | 2 | 0.566 | 0.569 | 0.652 | 0.557 | 0.517 | 0.522 | 0.492 |
| | | 4 | 1.005 | 0.944 | 1.048 | 1.006 | 0.942 | 0.893 | 0.908 |
| | | | Cetuximab (ug/mL) | | | | | | |
| RKO | ISC-4 | 1 | 0.235 | 0.265 | 0.265 | 0.37 | 0.31 | 0.294 | 0.258 |
| | (uM) | 2 | 0.47 | 0.508 | 0.513 | 0.442 | 0.487 | 0.464 | 0.44 |
| | | 4 | 0.639 | 0.668 | 0.667 | 0.64 | 0.634 | 0.607 | 0.646 |

To evaluate this combinatorial activity in one type of clinically relevant setting, the synergistic efficacy of ISC-4 and cetuximab is tested in RKO clones with evolved resistance to 5-FU. The synergistic activity of ISC-4 and cetuximab is retained despite acquired 5-FU-resistance in the colon cancer cells as shown in FIGS. 3E and 3F. FIG. 3E shows results of a cell viability assay of wild-type and 5-FU-resistant RKO cells treated with 5-FU as indicated for 24 hours (n=3). FIG. 3F shows results of 5-FU-resistant RKO cells treated with ISC-4 (2 μM) and cetuximab (1 μg/mL) for 24 hours (n=3).

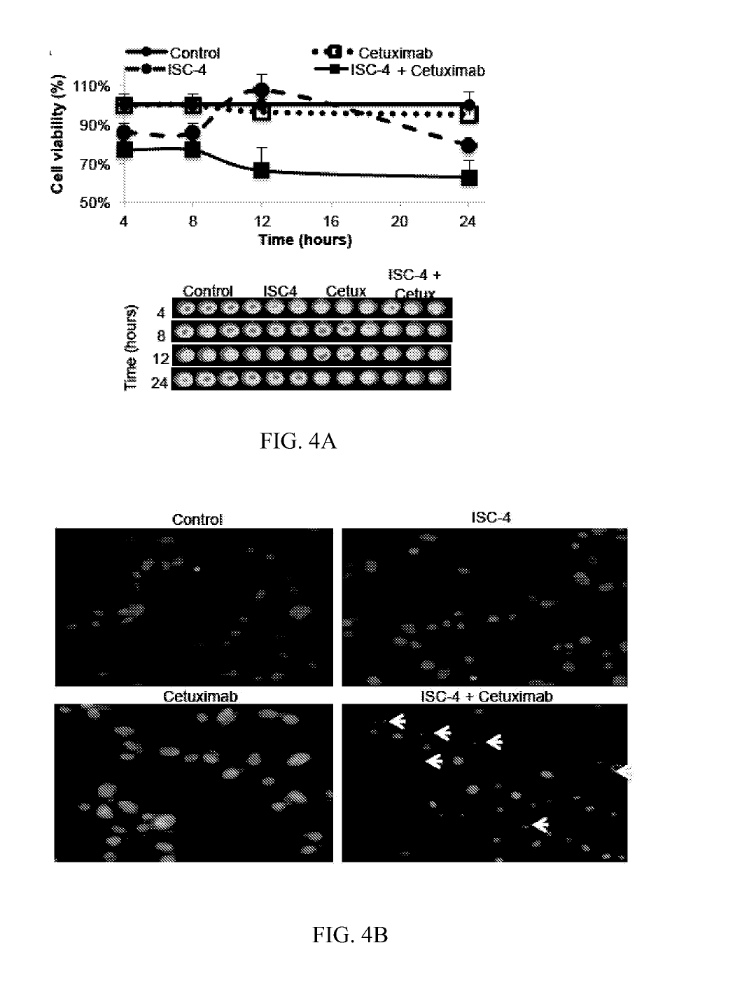

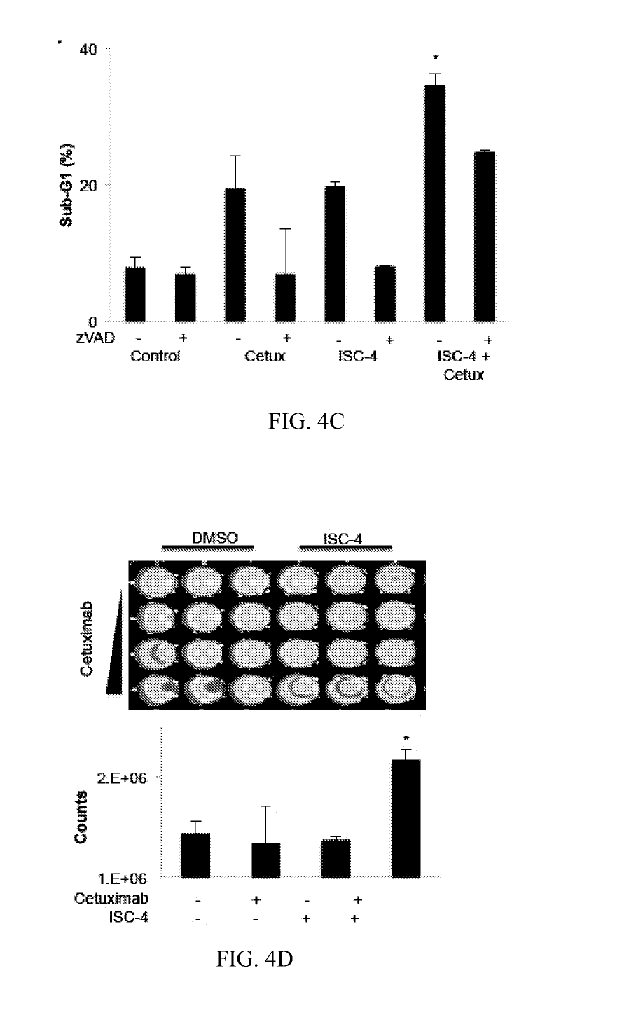

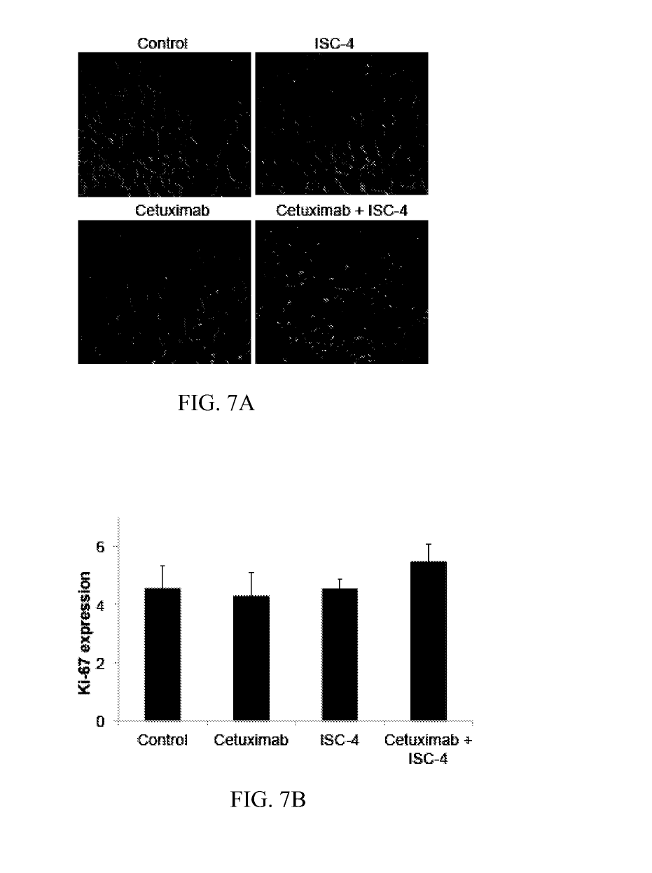

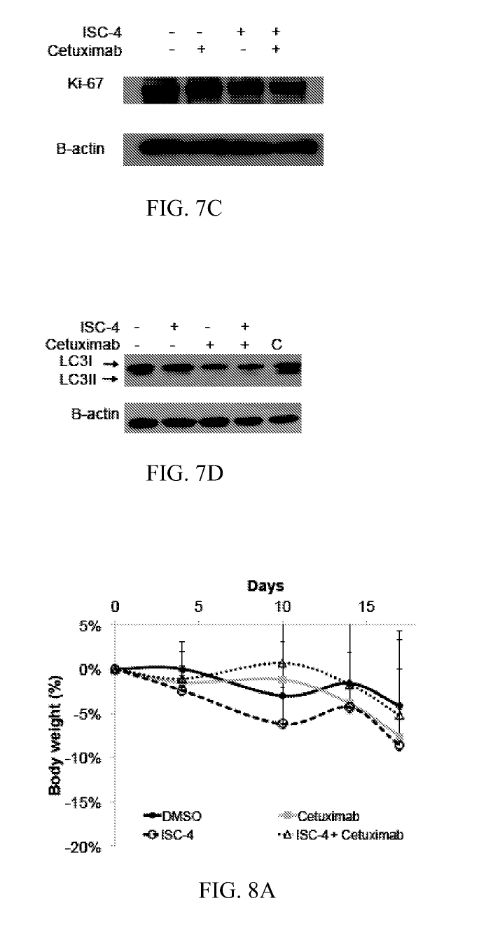

The kinetics of the synergistic efficacy of ISC-4 and cetuximab treatment is determined and such activity is found as early as 8 hours post-treatment, with greater synergy at 12 hours as shown in FIG. 4A. FIG. 4A shows results of cell viability assays of RKO cells treated with ISC-4 (2 μM) and cetuximab (1 μg/mL) alone or in combination for the indicated time period (n=3). This observation suggests that the synergistic activity of ISC-4 and cetuximab is perhaps cytotoxic rather than cytostatic. Changes in cell morphology, as well as fluorescent labeling of DNA, of treated cancer cells revealed that the ISC-4 and cetuximab combination treatment causes apparent DNA fragmentation as shown in FIG. 4B and FIG. 7A. FIG. 4B shows results of DAPI staining of RKO cells treated as in FIG. 4A for 12 hours. White arrows indicate cells with fragmented DNA. FIG. 7A shows phase-contrast microscopy of RKO cells treated with ISC-4 (2 μM) and cetuximab (1 μg/mL) alone or in combination for 12 hours.

Further analysis revealed that the combination of ISC-4 and cetuximab cooperatively and significantly increase sub-G1 content compared to either ISC-4 alone or cetuximab alone, but the combinatorial sub-G1 content is not sufficient to fully explain the observed synergy as shown in FIG. 4C. FIG. 4C shows sub-G1 content of RKO cells treated with ISC-4 (2 μM) and cetuximab (1 μg/mL) alone or in combination for 12 hours (n=3). *P<0.05 compared to all treatment groups by Student's two-tailed t test. ISC-4-induced sub-G1 content is significantly inhibited by co-incubation with the pan-caspase inhibitor zVAD-fmk, suggesting that the combination of ISC-4 and cetuximab induces caspase-dependent apoptosis. In support of this observation, the combination of ISC-4 and cetuximab synergistically induces caspase-3 activation as shown in FIG. 4D. FIG. 4D shows results of Caspase-Glo assay of RKO cells treated with ISC-4 (2 uM) in combination with cetuximab (0, 0.25, 0.5, or 1 μg/mL) at 24 hours post-treatment. The bottom panel of FIG. 4D shows quantification of ISC-4 (2 uM) and cetuximab (1 μg/mL) (n=3).

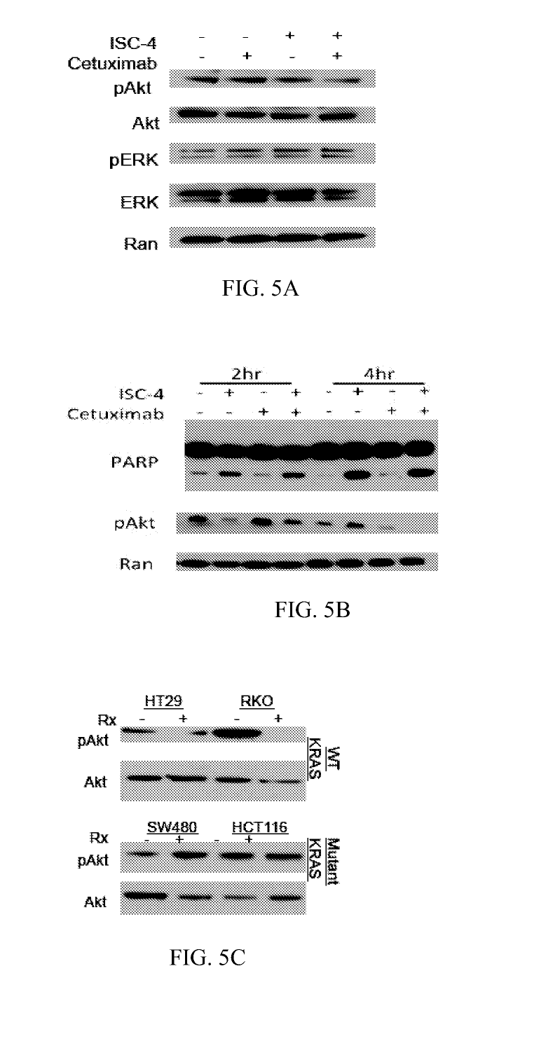

Western blot analysis reveals that ISC-4 in combination with cetuximab cooperatively reduces phospho-Akt levels, but not phospho-ERK, to a very modest level at 24 hours post-treatment as shown in FIG. 5A. FIG. 5A shows results of Western blot analysis of RKO cells treated with ISC-4 (2 μM) and cetuximab (1 μg/mL) alone or in combination for 24 hours. A time course analysis reveals that the combination cooperatively ablates phospho-Akt levels as soon as 4 hours post-treatment as shown in FIG. 5B. FIG. 5B shows results of Western blot analysis of RKO cells treated with ISC-4 (2 μM) and cetuximab (1 μg/mL) alone or in combination for indicated time periods. Ran is shown as a loading control. Human colon cancer cell lines that exhibit a synergistic response to ISC-4 and cetuximab also respond with a significant decrease in phospho-Akt as shown in FIG. 5C. FIG. 5C shows results of Western blot analysis of indicated human colon cancer cell lines following treatment with the combination (Rx) of ISC-4 (2 μM) and cetuximab (1 μg/mL) for 8 hrs. *P<0.05 compared to control.

Human colon cancer cell lines harboring mutant KRAS that did not respond synergistically to the combination therapy also did not exhibit any changes in phospho-Akt levels in response to treatment. Thus, phospho-Akt levels correlate with the antitumor response to ISC-4 and cetuximab.

No effect on Ki-67 expression or LC3B cleavage, a marker of autophagy, is observed with the combination as shown in FIGS. 7B, 7C and 7D. FIG. 7B shows results of flow cytometry analysis of Ki-67 expression in RKO cells treated with ISC-4 (2 μM) and cetuximab (1 μg/mL) alone or in combination. FIG. 7C shows Western bot analysis of Ki-67 expression in RKO cells treated with ISC-4 (2 μM) and cetuximab (1 μg/mL) alone or in combination. FIG. 7D shows results of Western blot analysis of RKO cells treated with ISC-4 (2 μM) and cetuximab (1 μg/mL) alone or in combination for 24 hours. Chloroquine (C; 10 μM) is included as a positive control for autophagy. Beta actin is shown as a loading control.

These observations indicate that combining ISC-4 with cetuximab leads to a cooperative decrease in phospho-Akt and cell viability, which are accompanied by increased apoptosis.

ISC-4 and Cetuximab Exert Synergistic Anti-Tumor Effects without Toxicity In Vivo

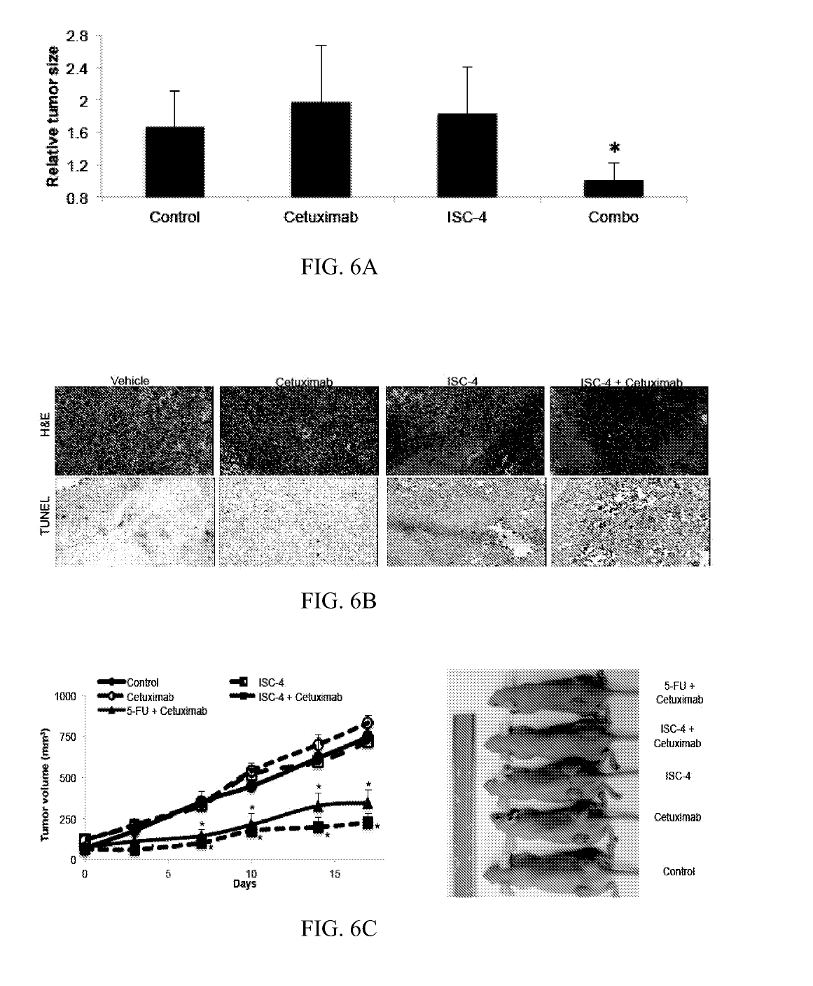

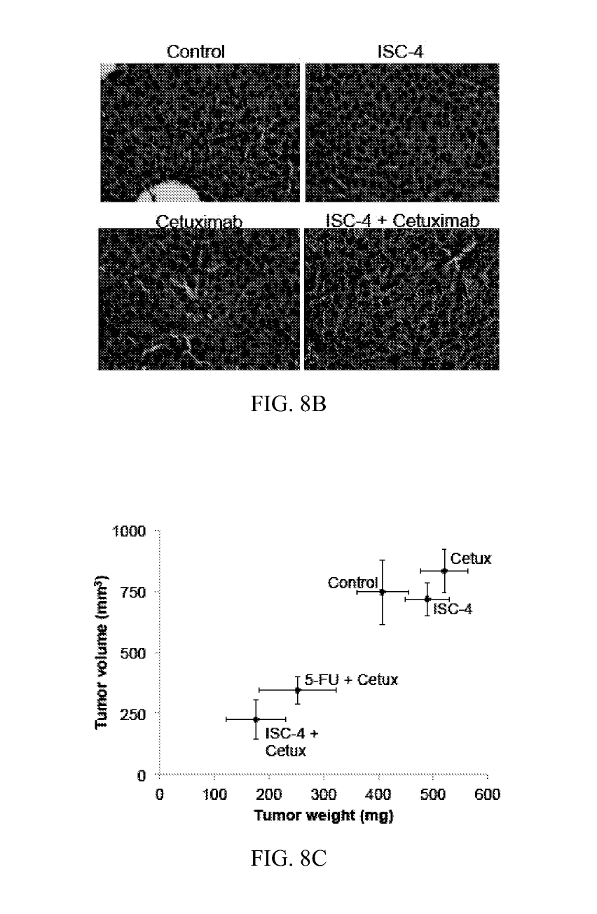

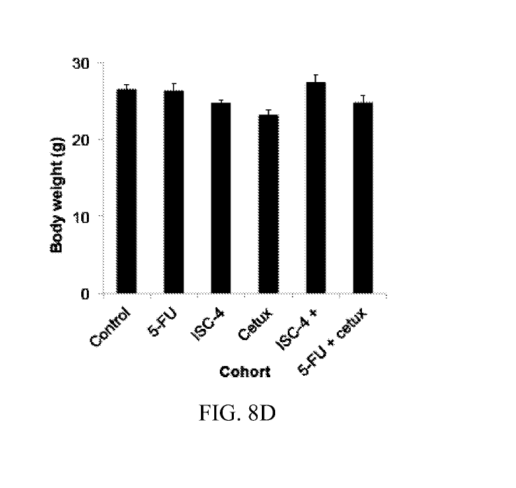

The anti-tumor efficacy of ISC-4 is tested in combination with cetuximab in advanced 5-FU-resistant RKO subcutaneous xenografts in this example. The combination therapy of ISC-4 and cetuximab has a synergistic initial effect on tumor progression and causes tumor stasis for the first week of therapy as shown in FIG. 6A. FIG. 6A shows relative tumor sizes of 5-FU-resistant RKO xenografts at 4 days post-treatment with a single dose of ISC-4 (3 mg/kg, i.p.), cetuximab (10 mg/kg, i.v.), or the combination (“combo”) (n≧5). Individual tumors were normalized to their baseline size measured on day 0, *P<0.05 compared to all treated groups using Student's two-tailed t test. Tissue analysis reveals that xenografts receiving the combination therapy of ISC-4 and cetuximab have higher levels of necrosis by histology and apoptosis by TUNEL staining than xenografts receiving either ISC-4 alone or cetuximab alone as shown in FIG. 6B. FIG. 6B shows results of hematoxylin and eosin (H&E) staining and TUNEL staining of xenograft tumors harvested 24 hours after treatment. The therapeutic dosing regimen employed in these studies is well tolerated and does not alter mouse body weight or change in liver histology as shown in FIGS. 8A and 8B. FIG. 8A shows change in body weight of mice receiving ISC-4 (3 mg/kg, i.p.), cetuximab (10 mg/kg, i.v.), or the combination (n≧5) twice a week for 2 weeks. Body weight changes are expressed relative to the body weight of each individual mouse prior to treatment on day 0 (n≧3). FIG. 8B shows results of H&E staining of liver tissue harvested from mice at 24 hours post-treatment with ISC-4 (3 mg/kg, i.p.), cetuximab (10 mg/kg, i.v.), or the combination.

The combination of ISC-4 and cetuximab in HT-29 xenografts in mice is examined in comparison with ISC-4 alone, cetuximab alone and the combination of cetuximab and 5-FU. Treatment with ISC-4 and cetuximab in combination strongly reduces tumor progression when given as weekly intravenous doses as is grossly apparent by tumor volume and tumor weight measurements, unlike ISC-4 alone or cetuximab alone, as shown in FIG. 6C and FIG. 8C. FIG. 6C shows results of treatment of athymic female nude mice harboring established HT-29 xenograft tumors with ISC-4 (3 mg/kg, i.v.), cetuximab (10 mg/kg, i.v.), the combination, or cetuximab and 5-FU (25 mg/kg, i.v.) once per week starting on day 0 (n≧8), error bars indicate SEM of replicates, *P<0.05 compared to control. FIG. 8C shows terminal tumor volume and tumor weight for HT-29 xenograft described in FIG. 6C. Treatment cohorts included ISC-4 (3 mg/kg, i.v.), cetuximab (10 mg/kg, i.v.), the combination, or cetuximab and 5-FU (25 mg/kg, i.v.) once per week (n≧8).

The combination of ISC-4 and cetuximab exhibits superior antitumor activity compared to the combination of 5-FU and cetuximab. The combination treatment of ISC-4 and cetuximab is well tolerated as shown in FIG. 8D. FIG. 8D shows mouse body weight at endpoint, which was three days following the last dose (n≧8), error bars indicate SEM of replicates. Serum chemistry analysis reveals no significant changes in electrolytes, liver function, or other molecular markers related to kidney or cardiac toxicity with chronic dosing as shown in Table IV.

Table IV shows serum chemistry profiles of mice receiving ISC-4 and cetuximab combination therapy. Athymic, female 8-week old nude mice received ISC-4 (3 mg/kg, i.p.), cetuximab (10 mg/kg, i.v.), or the combination (n≧5) twice a week for 2 weeks. Serum was collected 2 days following the last dose.

TABLE IV

| | | | | | | | | Alk. | | | |
|---|---|---|---|---|---|---|---|---|---|---|---|
| | Na | K | Cl | T. Bil | Creatinine | BUN | Glucose | Phos. | LDH | AST | ALT |
| Cohort | mmol/L | mmol/L | mmol/L | mg/dl | mg/dl | mg/dl | mg/dl | U/L | U/L | U/L | U/L |
| Control | 151 ± 0 | 5.1 ± 0 | 106 ± 0 | 0.85 ± 0.21 | 0.1 ± 0 | 23 ± 0 | 303 ± 1 | 49 ± 1 | 1183 ± 116 | 253 ± 11 | 29 ± 0 |
| ISC-4 | 155 ± 2 | 4.8 ± .1 | 107 ± 3 | 0.65 ± 0.07 | 0.1 ± 0 | 22 ± 1 | 309 ± 8 | 35 ± 10 | 1074 ± 531 | 221 ± 138 | 29 ± 2 |
| Cetux | 154 ± 2 | 5.8 ± .4 | 104 ± 5 | 0.733 ± 0.06 | 0.1 ± 0 | 21 ± 6 | 201 ± 48 | 56 ± 20 | 1183 ± 488 | 219 ± 22 | 27 ± 2 |
| ISC-4 + | 156 ± 0 | 6.5 ± 1 | 106 ± 1 | 0.95 ± 0.35 | 0.1 ± 0 | 29 ± 2 | 248 ± 19 | 53 ± 6 | 1240 ± 35 | 174 ± 28 | 21 ± 0 |
| Cetux | | | | | | | | | | | |

T. Bil, total bilirubin;
BUN, blood urea nitrogen,
Alk. Phos., alkaline phosphatase;
LDH, lactate dehydrogenase;
AST aspartate transminase;
ALT, alanine transaminase

Any patents or publications mentioned in this specification are incorporated herein by reference to the same extent as if each individual publication is specifically and individually indicated to be incorporated by reference.

Item List 1

Item 1: A method of treating cancer in a subject in need thereof, comprising: administering a combination of cetuximab and ISC-4 as a combination formulation or separately, wherein administration of the combination provides a synergistic effect.

Item 2: The method of treating cancer of item 1, wherein the cancer is characterized by wild-type KRAS.

Item 3: The method of treating cancer of item 1 or item 2, wherein the cancer is colorectal cancer characterized by wild-type KRAS.

Item 4: The method of treating cancer of any of items 1-3, further comprising: obtaining a first sample containing or suspected of containing cancer cells from the subject prior to administering the combination of cetuximab and ISC-4; obtaining a second sample containing or suspected of containing cancer cells from the subject after administering the combination of cetuximab and ISC-4; and assaying the first and second samples for one or more markers of apoptosis, thereby monitoring effectiveness of administering the combination of cetuximab and ISC-4.

Item 5: The method of treating cancer of any of items 1-3, further comprising: obtaining a first sample containing or suspected of containing cancer cells from the subject prior to administering the combination of cetuximab and ISC-4; obtaining a second sample containing or suspected of containing cancer cells from the subject after administering the combination of cetuximab and ISC-4; and assaying the first and second samples for phospho-Akt, thereby monitoring effectiveness of administering the combination of cetuximab and ISC-4.

Item 6: The method of treating cancer of any of items 1-5, wherein the cetuximab and ISC-4 are administered simultaneously.

Item 7: The method of treating cancer of any of items 1-5, wherein the cetuximab and ISC-4 are administered sequentially.

Item 8: The method of treating cancer of item 7, wherein the cetuximab and ISC-4 are administered sequentially within a period of time selected from: one hour, two hours, four hours, eight hours, twelve hours and twenty-four hours.

Item 9: A pharmaceutical composition comprising cetuximab and ISC-4.

Item 10: A commercial package comprising cetuximab and ISC-4.

Item 11: The commercial package of item 10, wherein the cetuximab and ISC-4 are provided as a single pharmaceutical formulation.

Item 12: The commercial package of item 10, wherein the cetuximab and ISC-4 are provided as separate pharmaceutical formulations.

Item 13: A method of treating cancer in a subject substantially as described herein.

Item 14: A pharmaceutical composition substantially as described herein

Item 15: A commercial package substantially as described herein.

Item 16: A method of treating cancer in a subject in need thereof, comprising: administering a combination of cetuximab and an ISC-4 prodrug as a combination formulation or separately, wherein administration of the combination provides a synergistic effect.

Item 17: The method of treating cancer of item 16, wherein the cancer is characterized by wild-type KRAS.

Item 18: The method of treating cancer of item 16 or 17, wherein the cancer is colorectal cancer characterized by wild-type KRAS.

Item 19: The method of treating cancer of any of items 16-18, further comprising: obtaining a first sample containing or suspected of containing cancer cells from the subject prior to administering the combination of cetuximab and ISC-4 prodrug; obtaining a second sample containing or suspected of containing cancer cells from the subject after administering the combination of cetuximab and ISC-4 prodrug; and assaying the first and second samples for one or more markers of apoptosis, thereby monitoring effectiveness of administering the combination of cetuximab and ISC-4 prodrug.

Item 20: The method of treating cancer of any of items 16-18, further comprising: obtaining a first sample containing or suspected of containing cancer cells from the subject prior to administering the combination of cetuximab and ISC-4 prodrug; obtaining a second sample containing or suspected of containing cancer cells from the subject after administering the combination of cetuximab and ISC-4 prodrug; and assaying the first and second samples for phospho-Akt, thereby monitoring effectiveness of administering the combination of cetuximab and ISC-4 prodrug.

Item 21: The method of treating cancer of any of items 16-20, wherein the cetuximab and ISC-4 prodrug are administered simultaneously.

Item 22: The method of treating cancer of any of items 16-20, wherein the cetuximab and ISC-4 prodrug are administered sequentially.

Item 23: The method of treating cancer of item 22, wherein the cetuximab and ISC-4 prodrug are administered sequentially within a period of time selected from: one hour, two hours, four hours, eight hours, twelve hours and twenty-four hours.

Item 24: The method of treating cancer of any of items 16-23, wherein the ISC-4 prodrug is ISC-4 glucosinolate prodrug having the structural formula:

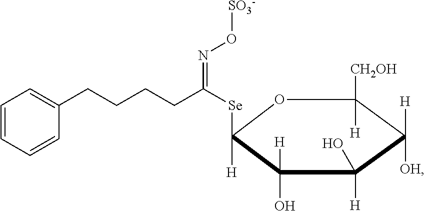

or a pharmaceutically acceptable salt thereof

Item 25: A pharmaceutical composition comprising cetuximab and ISC-4 glucosinolate prodrug having the structural formula:

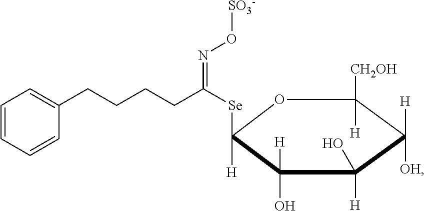

or a pharmaceutically acceptable salt thereof

Item 26: A commercial package comprising cetuximab and ISC-4 glucosinolate prodrug having the structural formula:

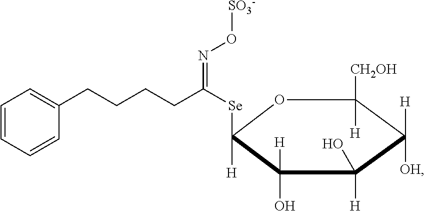

or a pharmaceutically acceptable salt thereof

Item 27: The commercial package of item 26, wherein the cetuximab and the ISC-4 glucosinolate prodrug or a pharmaceutically acceptable salt thereof, are provided as a single pharmaceutical formulation.

Item 28: The commercial package of item 26, wherein the cetuximab and the ISC-4 glucosinolate prodrug or a pharmaceutically acceptable salt thereof, are provided as separate pharmaceutical formulations.

Item 29: A composition comprising: ISC-4 glucosinolate prodrug having the structural formula:

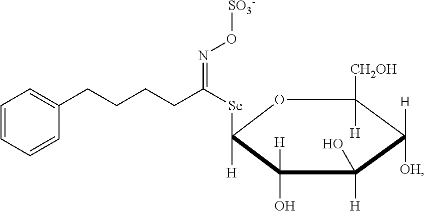

or a pharmaceutically acceptable salt thereof

Item List 2

Item 30: A method of treating cancer in a subject in need thereof, comprising: administering a combination of cetuximab and ISC-4 as a combination formulation or separately.

Item 31: The method of claim 30, wherein administration of the combination provides a synergistic effect.

Item 32: The method of treating cancer of claim 30 or 31, wherein the cancer is characterized by wild-type KRAS.

Item 33: The method of treating cancer of any of claims 30-32, wherein the cancer is colorectal cancer characterized by wild-type KRAS.

Item 34: The method of treating cancer of any of claims 30-33, further comprising: obtaining a first sample containing or suspected of containing cancer cells from the subject prior to administering the combination of cetuximab and ISC-4; obtaining a second sample containing or suspected of containing cancer cells from the subject after administering the combination of cetuximab and ISC-4; and assaying the first and second samples for one or more markers of apoptosis, thereby monitoring effectiveness of administering the combination of cetuximab and ISC-4.

Item 35: The method of treating cancer of any of claims 30-34, further comprising: obtaining a first sample containing or suspected of containing cancer cells from the subject prior to administering the combination of cetuximab and ISC-4; obtaining a second sample containing or suspected of containing cancer cells from the subject after administering the combination of cetuximab and ISC-4; and assaying the first and second samples for phospho-Akt, thereby monitoring effectiveness of administering the combination of cetuximab and ISC-4.

Item 36: The method of treating cancer of any of claims 30-35, wherein the cetuximab and ISC-4 are administered simultaneously.

Item 37: The method of treating cancer of any of claims 30-35, wherein the cetuximab and ISC-4 are administered sequentially.

Item 38: The method of treating cancer of any of claims 30-35 and 37, wherein the cetuximab and ISC-4 are administered sequentially within a period of time selected from: one hour, two hours, four hours, eight hours, twelve hours and twenty-four hours.

Item 39: A method of treating cancer in a subject in need thereof, comprising: administering a combination of cetuximab and an ISC-4 prodrug as a combination formulation or separately.

Item 40: The method of treating cancer of claim 39, wherein administration of the combination provides a synergistic effect.

Item 41: The method of treating cancer of any of claim 39 or 40, wherein the cancer is characterized by wild-type KRAS.

Item 42: The method of treating cancer of any of claims 39-41, wherein the cancer is colorectal cancer characterized by wild-type KRAS.

Item 43: The method of treating cancer of any of claims 39-42, further comprising: obtaining a first sample containing or suspected of containing cancer cells from the subject prior to administering the combination of cetuximab and the ISC-4 prodrug; obtaining a second sample containing or suspected of containing cancer cells from the subject after administering the combination of cetuximab and the ISC-4 prodrug; and assaying the first and second samples for one or more markers of apoptosis, thereby monitoring effectiveness of administering the combination of cetuximab and the ISC-4 prodrug.

Item 44: The method of treating cancer of any of claims 39-43, further comprising: obtaining a first sample containing or suspected of containing cancer cells from the subject prior to administering the combination of cetuximab and the ISC-4 prodrug; obtaining a second sample containing or suspected of containing cancer cells from the subject after administering the combination of cetuximab and the ISC-4 prodrug; and assaying the first and second samples for phospho-Akt, thereby monitoring effectiveness of administering the combination of cetuximab and the ISC-4 prodrug.

Item 45: The method of treating cancer of any of claims 39-44, wherein the cetuximab and the ISC-4 prodrug are administered simultaneously.

Item 46: The method of treating cancer of any of claims 39-44, wherein the cetuximab and the ISC-4 prodrug are administered sequentially.

Item 47: The method of treating cancer of any of claims 39-44 and 46, wherein the cetuximab and the ISC-4 prodrug are administered sequentially within a period of time selected from: one hour, two hours, four hours, eight hours, twelve hours and twenty-four hours.

Item 48: The method of treating cancer of any of claims 39-47, wherein the ISC-4 prodrug is ISC-4 glucosinolate prodrug having the structural formula:

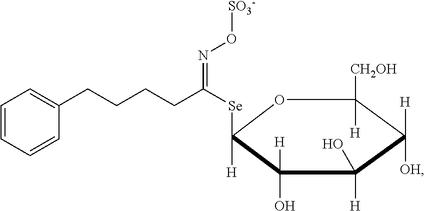

or a pharmaceutically acceptable salt thereof

Item 49: The method of treating cancer of any of claims 30-48, wherein the cancer is resistant to 5-fluorouracil.

Item 50: The method of treating cancer of any of claims 30-49, wherein the cancer is colorectal cancer resistant to 5-fluorouracil.

Item 51: The method of treating cancer of any of claims 30-50, wherein the cancer is resistant to 5-fluorouracil and characterized by wild-type KRAS.

Item 52: The method of treating cancer of any of claims 30-51, wherein the cancer is resistant to 5-fluorouracil and characterized by wild-type KRAS such that the wild-type KRAS does not have an activating KRAS mutation, in codon 12, 13 or 61, with reference to human KRAS.

Item 53: The method of treating cancer of any of claims 30-52, wherein the cancer is resistant to 5-fluorouracil and characterized by wild-type KRAS such that the wild-type KRAS does not have activating KRAS mutations Q61H, G12S, G12V, G12A or G13D, with reference to human KRAS.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A pharmaceutical composition comprising: cetuximab and ISC-4; or cetuximab and an ISC-4 prodrug.

2. The pharmaceutical composition of claim 1, wherein the ISC-4 prodrug is ISC-4 glucosinolate prodrug having the structural formula: 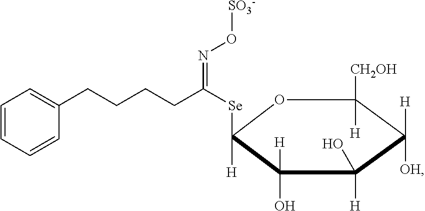
or a pharmaceutically acceptable salt thereof.

3. A commercial package comprising cetuximab and ISC-4; or cetuximab and an ISC-4 prodrug.

4. The commercial package of claim 3 wherein the ISC-4 prodrug is ISC-4 glucosinolate prodrug having the structural formula: 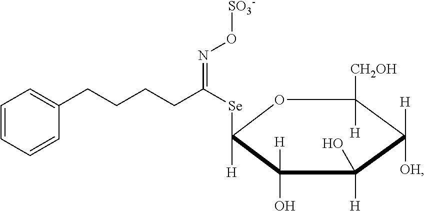
or a pharmaceutically acceptable salt thereof.

5. The commercial package of claim 3, wherein the cetuximab and ISC-4 are provided as a single pharmaceutical formulation.

6. The commercial package of claim 3, wherein the cetuximab and ISC-4 are provided as separate pharmaceutical formulations.

7. The commercial package of claim 3 or 4, wherein the cetuximab; and the ISC-4 prodrug are provided as a single pharmaceutical formulation.

8. The commercial package of claim 3 or 4, wherein the cetuximab; and the ISC-4 prodrug are provided as separate pharmaceutical formulations.

9. A composition comprising: ISC-4 glucosinolate prodrug having the structural formula: 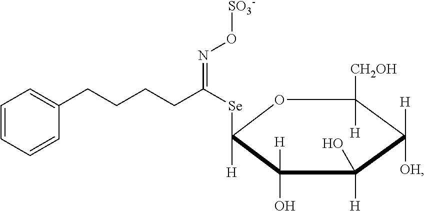
or a pharmaceutically acceptable salt thereof.

10. A method of treating cancer in a subject in need thereof, comprising:
administering a combination of cetuximab and ISC-4 as a combination formulation or separately.

11. The method of claim 10, wherein administration of the combination provides a synergistic effect.

12. The method of treating cancer of claim 10 or 11, wherein the cancer is characterized by wild-type KRAS.

13. The method of treating cancer of any of claims 10-12, wherein the cancer is colorectal cancer characterized by wild-type KRAS.

14. The method of treating cancer of any of claims 10-13, further comprising:
obtaining a first sample containing or suspected of containing cancer cells from the subject prior to administering the combination of cetuximab and ISC-4;
obtaining a second sample containing or suspected of containing cancer cells from the subject after administering the combination of cetuximab and ISC-4; and
assaying the first and second samples for one or more markers of apoptosis, thereby monitoring effectiveness of administering the combination of cetuximab and ISC-4.

15. The method of treating cancer of any of claims 10-14, further comprising:
obtaining a first sample containing or suspected of containing cancer cells from the subject prior to administering the combination of cetuximab and ISC-4;
obtaining a second sample containing or suspected of containing cancer cells from the subject after administering the combination of cetuximab and ISC-4; and
assaying the first and second samples for phospho-Akt, thereby monitoring effectiveness of administering the combination of cetuximab and ISC-4.

16. The method of treating cancer of any of claims 10-15, wherein the cetuximab and ISC-4 are administered simultaneously.

17. The method of treating cancer of any of claims 10-15, wherein the cetuximab and ISC-4 are administered sequentially.

18. The method of treating cancer of any of claims 10-15 and 17, wherein the cetuximab and ISC-4 are administered sequentially within a period of time selected from: one hour, two hours, four hours, eight hours, twelve hours and twenty-four hours.

19. A method of treating cancer in a subject in need thereof, comprising:
administering a combination of cetuximab and an ISC-4 prodrug as a combination formulation or separately.

20. The method of treating cancer of claim 19, wherein administration of the combination provides a synergistic effect.

21. The method of treating cancer of claim 19 or 20, wherein the cancer is characterized by wild-type KRAS.

22. The method of treating cancer of any of claims 19-21, wherein the cancer is colorectal cancer characterized by wild-type KRAS.

23. The method of treating cancer of any of claims 19-22, further comprising:
obtaining a first sample containing or suspected of containing cancer cells from the subject prior to administering the combination of cetuximab and ISC-4 prodrug;
obtaining a second sample containing or suspected of containing cancer cells from the subject after administering the combination of cetuximab and ISC-4 prodrug; and
assaying the first and second samples for one or more markers of apoptosis, thereby monitoring effectiveness of administering the combination of cetuximab and ISC-4 prodrug.

24. The method of treating cancer of any of claims 19-23, further comprising:
obtaining a first sample containing or suspected of containing cancer cells from the subject prior to administering the combination of cetuximab and ISC-4 prodrug;
obtaining a second sample containing or suspected of containing cancer cells from the subject after administering the combination of cetuximab and ISC-4 prodrug; and
assaying the first and second samples for phospho-Akt, thereby monitoring effectiveness of administering the combination of cetuximab and ISC-4 prodrug.

25. The method of treating cancer of any of claims 19-24, wherein the cetuximab and ISC-4 prodrug are administered simultaneously.

26. The method of treating cancer of any of claims 19-24, wherein the cetuximab and ISC-4 prodrug are administered sequentially.

27. The method of treating cancer of any of claims 19-24 and 26, wherein the cetuximab and ISC-4 prodrug are administered sequentially within a period of time selected from: one hour, two hours, four hours, eight hours, twelve hours and twenty-four hours.

28. The method of treating cancer of any of claims 19-27, wherein the ISC-4 prodrug is ISC-4 glucosinolate prodrug having the structural formula: 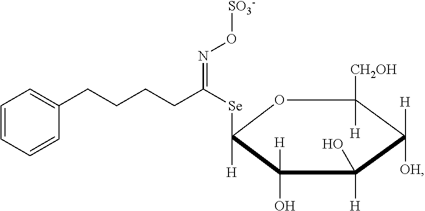
or a pharmaceutically acceptable salt thereof.

29. The method of treating cancer of any of claims 10-28, wherein the cancer is resistant to 5-fluorouracil.

30. The method of treating cancer of any of claims 10-29, wherein the cancer is resistant to 5-fluorouracil and characterized by wild-type KRAS.

31. The method of treating cancer of any of claims 10-30, wherein the cancer is resistant to 5-fluorouracil and characterized by wild-type KRAS such that the wild-type KRAS does not have an activating KRAS mutation, in codon 12, 13 or 61, with reference to human KRAS.

32. The method of treating cancer of any of claims 10-31, wherein the cancer is resistant to 5-fluorouracil and characterized by wild-type KRAS such that the wild-type KRAS does not have activating KRAS mutations Q61H, G12S, G12V, G12A or G13D, with reference to human KRAS.

33. Cetuximab and ISC-4 or cetuximab and an ISC-4 prodrug for use in the treatment of cancer.

34. A combination of cetuximab and ISC-4 or cetuximab and an ISC-4 prodrug for use as a medicament.

35. A method of treating cancer in a subject substantially as described herein.

36. A pharmaceutical composition substantially as described herein

37. A commercial package substantially as described herein.
